(19) United States
(12) Patent Application Publication
(10) Pub. No.: US 2014/0317777 A1
(43) Pub. Date: Oct. 23, 2014

(54) CASSAVA

(71) Applicant: Donald Danforth Plant Science Center, St. Louis, MO (US)

(72) Inventors: Richard Sayre, Los Alamos, NM (US); Narayanan Narayanan, St. Louis, MO (US)

(73) Assignee: Donald Danforth Plant Science Center, St. Louis, MO (US)

(21) Appl. No.: 14/112,004

(22) PCT Filed: Apr. 12, 2012
(86) PCT No.: PCT/US12/33337

§ 371 Date: Jan. 20, 2014

Related U.S. Application Data
(60) Provisional application No. 61/474,818, filed on Apr. 13, 2011.

(51) Int. Cl. C12N 15/82

(52) U.S. Cl. CPC C12N 15/8251; C12N 15/8243
USPC 800/278; 800/298

(57) ABSTRACT

Provided are methods for reducing levels of cyanogenic glycosides and improving protein content in plants, as well as plants containing reduced levels of cyanogenic glycosides and improved protein content. In one aspect, such methods comprise, and such plants are created via, tissue-specific expression of a storage protein such as hydroxynitrile lyase in the apoplastic space of cells of the roots and tubers of such plants.

### CROSS REFERENCE TO RELATED APPLICATIONS

This application claims the benefit of priority of U.S. Provisional Application Ser. No. 61/474,818, filed Apr. 13, 2011, the contents of which are herein incorporated by reference in their entirety.

### STATEMENT REGARDING FEDERALLY SPONSORED RESEARCH OR DEVELOPMENT

Not applicable

### BACKGROUND OF THE INVENTION

### Field of the Invention

The present invention relates to methods and transgenic plants that exhibit reduced levels of cyanogenic glycosides and improved protein content while accelerating cyanogen turnover during root processing. In one aspect, such methods and transgenic plants are created through the tissue-specific expression of hydroxynitrile lyase in the roots and root storage cells of the plants.

More than 3,000-12,000 plant species, including many important crops such as cassava, sorghum, flax, almonds, lima beans, and white clover, produce potentially toxic levels of cyanogenic glucosides, a group of nitrile containing plant secondary compounds yielding cyanide upon enzymatic breakdown (Hickel et al., (1996) Physiol. Plant. 98: 891-898; Wajant and Effenberger, (1996) J. Biol. Chem. 377: 611-617; hakes, (1985) Planta 166: 156-160).

Cassava (Mannihot esculenta), for example, plays a significant role in the economic productivity of many developing countries, and especially those in Sub-Saharan Africa, because of its ability to grow in poor soils, under low rainfall conditions, and its amenability to harvesting throughout the year. Additionally, its wide harvesting window allows it to act as a famine reserve, and offers flexibility to resource-poor farmers (Stone, (2002) Curr. Anthropol. 43: 611-630). Though cassava is a major source of dietary carbohydrates for more than 500 million people worldwide and 250 million people in Sub-Saharan Africa, it is a poor source of protein, and lacks many essential micronutrients and vitamins. Cassava has the lowest protein-to-energy ratio of any staple food crop in the world. Since a diet based primarily on cassava provides less than 30% of the minimum daily requirement for protein, additional food sources are required to ensure a balanced diet (Cock, (1985) Cassava: new potential for a neglected crop. Westview press. Boulder, Colo. pp. 191).

Moreover, cassava contains potentially toxic levels of cyanogenic glucosides such as linamarin (95%) and lotaustralin (5%) in all parts of the plant except seeds (Conn, (1994) Acta Hort. 375: 31-43). Leaves have high cyanogenic glucoside levels (5 g linamarin/kg fresh weight), whereas roots have approximately 20 fold lower levels (White et al., (1998) Plant Physiol. 116: 1219-1225). Various health disorders have been associated with the consumption of cassava due to the residual levels of cyanogens. Chronic, low-level cyanide exposure is associated with the development of goiter and tropical ataxic neuropathy, a nerve-damaging disorder that renders a person unsteady and uncoordinated (Osuntokun, (1981) World Rev. Nutr. Diet. 36: 141-173). Severe cyanide poisoning, particularly during famines, is associated with outbreaks of a debilitating, irreversible paralytic disorder called Konzo and, in some cases, death (Tylleskar et al., (1992) Lancet. 339: 208-211). People with protein deficiency are particularly susceptible to cyanide poisoning as they lack the proper amino acids necessary to help detoxify the cyanide poison. On the other hand, these cyanogenic glycosides have been shown to protect cassava from herbivores and insects, as well as from theft (Nahrstedt, (1985) Plant Syst. Evol. 150: 35-47).

The physiology and the biochemical pathways of cyanogenesis in cassava have been previously investigated by several groups (McMahon et al., (1995) J. Exp. Bot. 46: 731-741; Siritunga and Sayre, (2007) JAOAC Int. 90: 1450-1455). Typically, cyanogenesis is initiated by rupturing of plant vacuoles, releasing linamarin which is hydrolyzed by linamarase, a cell wall-associated beta-glycosidase, after tissue injury. Hydrolysis of linamarin yields an unstable hydroxynitrile intermediate, acetone cyanohydrins, which can spontaneously or enzymatically decompose to form cyanide (McMahon et al., (1995) J. Exp. Bot. 46: 731-741; Pancoro and Hughes, (1992) Plant J. 2: 821-827; Santana et al., (2002) Plant Physiol. 129: 1686-1694). The enzymatic production of cyanide from acetone cyanohydrin is catalyzed by the enzyme hydroxynitrile lyase (HNL). Spontaneous production also occurs when the pH is greater than about 5.0, or at temperatures greater than about 35° C. (White et al., (1994) Plant Physiol. 116: 1219-1225; White et al., (1998) Acta Hort. 375: 69-78; Siritunga and Sayre, (2007) JAOAC Int. 90: 1450-1455).

It has been proposed that cassava plants may use linamarin as a transportable source of reduced nitrogen for amino acid synthesis in roots (Siritunga and Sayre, (2007) JAOAC Int. 90: 1450-1455; Jenrich et al., (2007) Proc. Natl. Acad. Sci. USA. 104: 18848-18853). Several reports suggest that linamarin is synthesized in cassava leaves and transported to roots (Siritunga and Sayre, (2004) Plant Mol. Biol. 56: 661-669, Jorgensen et al., (2005) Plant Physiol. 139: 363-374). Linamarin may then be deglycosylated by a β-glucosidase, generating cyanide, which can then be assimilated along with cysteine via β-cyanoalanine synthase to produce β-cyanoalanine and sulfide. Following hydration of β-cyanoalanine to form asparagine, deamination of this amino acid generates aspartate and free ammonia, which can then be re-assimilated by the glutamine synthetase/synthase cycle into other amino acids (Lea et al., (1992) In Nitrogen Metabolism of Plants (Proceedings of the Phytochemical Society of Europe, 33), K. Mengel and D. J. Pilbean, eds., Clarendon, Oxford, pp. 153-186). Consistent with this hypothesis, it has been shown that the activities of β-cyanoalanine synthase and β-cyanoalanine hydrase are 3-fold higher in roots when compared with that in cassava leaves (Elias et al., (1997a) Phytochem. 46: 469-472; Elias et al., (1997b) Plant Sci: 126: 155-162). It has also been shown that β-cyano-alanine hydrase from seedlings of lupine (Lupinus angustifolius) is a NIT4 (Arabidopsis thaliana nitrilase 4) orthologue (Piotrowski and Volmer, (2006) Plant Mol. Biol. 61: 111-122).

Several transgenic approaches have been attempted to reduce or eliminate cyanogenesis in crop plants, including cassava (Siritunga and Sayre, (2003) Planta, 217: 367-373; Sirtunga and Sayre, (2004) Plant Mol. Biol. 56: 661-669; and Jorgensen et al., (2005) Plant Physiol. 139: 363-374). Tissue-specific inhibition of the expression of the CYP79D1/D2 cytochrome P450 enzymes responsible for the first step of linamarin synthesis in leaves leads to 99% reduction in root cyanogen levels in cassava, suggesting that the linamarin is synthesized in leaves and transported to roots (Siritunga and Sayre, (2007) JAOAC Int. 90: 1450-1455). However, these transgenics plants had substantially impaired growth due to reduced levels of cyanogens for amino acid synthesis in the roots (Siritunga and Sayre, (2003) Planta, 217: 367-373). It is also well established that overexpression of HNL throughout all the tissues of a plant accelerates the conversion of acetone cyanohydrins to cyanide, and thereby facilitates the detoxification of cassava roots and cyanide volatilization during processing (Siritunga and Sayre, (2004) Plant Mol. Biol. 56: 661-669).

However, there remains the need to develop improved cassava varieties that exhibit significantly reduced root toxicity, yet maintain normal levels of linamarin in their leaves to protect against herbivores, and that additionally possess superior nutritional properties. In addition, acceleration of the conversion of cyanogens to cyanide in roots would allow for more efficient detoxification of cyanogens during root processing for commercial starch extraction since there are well developed technologies for cyanide mitigation.

The present invention meets these needs and is based, at least in part, upon the surprising discovery that tissue-specific expression of a storage protein and/or HNL in the apoplastic space of roots not only reduces cyanide levels, but also significantly increases protein synthesis and protein content throughout the transgenic cassava plants.

### SUMMARY OF THE INVENTION

In one embodiment, the invention includes a transgenic plant comprising a heterologous nucleic acid sequence comprising a promoter preferentially active in a root or storage organ that is operatively linked to a nucleic acid sequence encoding a storage protein. In one aspect, the transgenic plant is cassava. In one aspect, the storage protein is targeted to the apoplast. In one aspect, the storage protein is selected from the group consisting of hydroxynitrile lyase, arachins, avenins, cocosins, conarchins, concocosins, conglutins, conglycinins, convicines, crambins, cruciferins, cucurbitins, dioscorins, edestins, excelesins, gliadins, glutens, glytenins, glycinins, helianthins, hordeins, kafirins, legumins, napins, oryzins, pennisetins, phaseolins, prolamines, psophocarpins, secalins, sporamins, tryspsin inhibitors, vicilins, vicines, and zeins.

In one aspect, the transgenic plant is characterized by the ability to form tubers having a protein content of at least 30 μg/mg based on the (dry substance) weight of the tuber.

In one aspect, the promoter drives expression of the storage protein substantially exclusively (predominantly) in the root of the transgenic plant. In one aspect, the promoter is a potato class I patatin promoter. In one aspect, the promoter has at least 80% nucleotide sequence identity to a class I patatin promoter (SEQ ID NO:13). In one aspect, the promoter has at least 90% nucleotide sequence identity to a class I patatin promoter (SEQ ID NO:13). In one aspect, the promoter has at least 95% nucleotide sequence identity to a class I patatin promoter (SEQ ID NO: 13). In one aspect, the promoter comprises SEQ ID NO:13.

In one aspect, the storage protein is a hydroxynitrile lyase. In one aspect, the hydroxynitrile lyase is from Manihot esculenta, Hevea Brasiliensis, or Baliospermum montanum. In one aspect, the hydroxynitrile lyase is from Manihot esculenta. In one aspect, the hydroxynitrile lyase has at least 90% amino acid sequence identity to SEQ ID NO:15. In one aspect, the hydroxynitrile lyase has at least 95% amino acid sequence identity to SEQ ID NO:15.

In another embodiment, the current invention includes a method of increasing the protein content of plant that produces cyanogenic glucosides, said method comprising:
i) transforming the plant with at least one nucleic acid molecule comprising a promoter preferentially active in a root or storage organ operatively linked to at least one transgene that encodes a storage protein, to produce a transformed plant;
ii) selecting the transformed plant comprising at least one transgene;
iii) growing the transformed plant to produce a plant exhibiting increased protein content when compared to an equivalent non-transformed plant, wherein the transformed plant and the equivalent non-transformed plant are grown under similar conditions.

In one aspect, the storage protein is targeted to the apoplastic space. In one aspect, targeting is achieved via the use of targeting sequences from a native HNL or linamarase.

In one aspect, the storage protein is selected from the group consisting of hydroxynitrile lyases, arachins, avenins, cocosins, conarchins, concocosins, conglutins, conglycinins, convicines, crambins, cruciferins, cucurbitins, dioscorins, edestins, excelesins, gliadins, glutens, glytenins, glycinins, helianthins, hordeins, kafirins, legumins, napins, oryzins, pennisetins, phaseolins, prolamines, psophocarpins, secalins, sporamins, tryspsin inhibitors, vicilins, vicines, and zeins.

In one aspect, the promoter drives expression of the storage protein substantially exclusively in the root or tuber of the transgenic plant.

In one aspect, the plant is selected from the group consisting of Cassava (Manihot esculenta), Sorghum (Sorghum vulgare), Flax (Linum usitatissimum), Lima beans (Phaseolus lunatus), Giant taro (Alocasia macrorrhizos), Bamboo (Bambusa arundinacea), Apple (Malus spp.), Peach (Prunus persica), Nectarine (Prunus persica var nucipersica), Cherry (Prunus spp.), Bitter almond (Prunus dulcis), raspberry, and crabapple. In one aspect, the plant is Cassava (Manihot esculenta).

In one aspect, the storage protein is hydroxynitrile lyase. In one aspect, the hydroxynitrile lyase is from Manihot esculenta, Hevea Brasiliensis, or Baliospermum montanum. In one aspect, the hydroxynitrile lyase is from Manihot esculenta. In one aspect, the hydroxynitrile lyase has at least 80% amino acid sequence identity to SEQ ID NO:15. In one aspect, the hydroxynitrile lyase has at least 90% amino acid sequence identity to SEQ ID NO: 15. In one aspect, the hydroxynitrile lyase has at least 95% amino acid sequence identity to SEQ ID NO:15.

In one aspect, the promoter is from a potato class I patatin promoter. In one aspect, the promoter has at least 80% nucleotide sequence identity to a class I patatin promoter (SEQ ID NO:13). In one aspect, the promoter has at least 90% nucleotide sequence identity to a class I patatin promoter (SEQ ID NO:13). In one aspect, the promoter has at least 95% nucleotide sequence identity to a class I patatin promoter (SEQ ID NO:13). In one aspect, the promoter comprises SEQ ID NO:13. In one aspect, the promoter consists essentially of SEQ ID NO:13.

In one aspect, the invention includes a transgenic plant or progeny thereof, produced by any of the preceding methods. In one aspect, the invention includes a plant cell produced by any of the preceding methods. In one aspect, the invention includes a transgenic seed produced by any of the preceding methods.

In another embodiment, the current invention includes a method of decreasing the cyanide content of plant-derived products from a plant that produces cyanogenic glucosides, said method comprising:

i) transforming the plant with at least one nucleic acid molecule comprising a promoter preferentially active in a root or storage organ operatively linked to at least one transgene that encodes a plant hydroxynitrile lyase, to produce a transformed plant;
ii) selecting the transformed plant comprising the at least one transgene;
iii) growing the transformed plant to produce a plant exhibiting decreased cyanide content when compared to an equivalent non-transformed plant, wherein the transformed plant and the equivalent non-transformed plant are grown under similar conditions; and

wherein the promoter drives expression of the hydroxynitrile lyase substantially exclusively in the root or tuber of the transgenic plant.

In one aspect, the hydroxynitrile lyase is targeted to the apoplastic space. In one aspect, targeting is achieved via the use of targeting sequences from a native HNL or linamarase.

In one aspect, the plant is selected from the group consisting of Cassava (Manihot esculenta), Sorghum (Sorghum vulgare), Flax (Linum usitatissimum), Lima beans (Phaseolus lunatus), Giant taro (Alocasia macrorrhizos), Bamboo (Bambusa arundinacea), Apple (Malus spp.), Peach (Prunus persica), Nectarine (Prunus persica var nucipersica), Cherry (Prunus spp.), Bitter almond (Prunus dulcis), raspberry, and crabapple. In one aspect, the plant is Cassava.

In one aspect, the hydroxynitrile lyase is from Manihot esculenta, Hevea Brasiliensis, or Baliospermum montanum. In one aspect, the hydroxynitrile lyase is from Manihot esculenta.

In one aspect, the plant hydroxynitrile lyase has at least 80% amino acid sequence identity to SEQ ID NO:15. In one aspect, the plant hydroxynitrile lyase has at least 90% amino acid sequence identity to SEQ ID NO:15. In one aspect, the hydroxynitrile lyase has at least 95% amino acid sequence identity to SEQ ID NO:15.

In one aspect, the promoter is from a potato class I patatin promoter. In one aspect, the promoter has at least 80% nucleotide sequence identity to a class I patatin promoter (SEQ ID NO:13). In one aspect, the promoter has at least 90% nucleotide sequence identity to a class I patatin promoter (SEQ ID NO:13). In one aspect, the promoter has at least 95% nucleotide sequence identity to a class I patatin promoter (SEQ ID NO:13). In one aspect, the promoter comprises SEQ ID NO:13. In one aspect, the promoter consists essentially of SEQ ID NO:13.

In another embodiment, the current invention includes a vector comprising a promoter preferentially active in a root or storage organ operatively linked with at least one transgene, the transgene being operatively linked with a 3′ untranslated region, wherein said at least one transgene encodes a plant hydroxynitrile lyase.

In one aspect, the promoter drives expression of the hydroxynitrile lyase substantially exclusively in the root or tuber of the transgenic plant.

In one aspect, the hydroxynitrile lyase is operatively coupled to targeting sequences that direct secretion of the hydroxynitrile lyase to the apoplastic space. In one aspect, the hydroxynitrile lyase is from Manihot esculenta, Hevea Brasiliensis, or Baliospermum montanum. In one aspect, the hydroxynitrile lyase is from Manihot esculenta.

In one aspect, the hydroxynitrile lyase has at least 80% amino acid sequence identity to SEQ ID NO:15. In one aspect, the hydroxynitrile lyase has at least 90% amino acid sequence identity to SEQ ID NO:15. In one aspect, the hydroxynitrile lyase has at least 95% amino acid sequence identity to SEQ ID NO:15.

In one aspect, the promoter is from a potato class I patatin promoter. In one aspect, the promoter has at least 80% nucleotide sequence identity to a class I patatin promoter (SEQ ID NO:13).

In one aspect, the promoter has at least 90% nucleotide sequence identity to a class I patatin promoter (SEQ ID NO:13). In one aspect, the promoter has at least 95% nucleotide sequence identity to a class I patatin promoter (SEQ ID NO:13). In one aspect, the promoter comprises SEQ ID NO:13.

In another embodiment, the current invention includes a method for producing hydroxynitrile lyase from a transgenic plant that produces cyanogenic glucosides, comprising:
i) cultivating the transgenic plant, wherein the transgenic plant comprises a heterologous nucleic acid sequence comprising a promoter that is operatively linked to a nucleic acid sequence encoding hydroxynitrile lyase, and
   wherein the promoter drives expression of the hydroxynitrile lyase substantially exclusively in the root or tuber of the cassava plant; and
ii) isolating the hydroxynitrile lyase from the tubers of the cultivated plants.

In one aspect, the plant is selected from the group consisting of Cassava (Manihot esculenta), Sorghum (Sorghum vulgare), Flax (Linum usitatissimum), Lima beans (Phaseolus lunatus), Giant taro (Alocasia macrorrhizos), and Bamboo. In one aspect, the transgenic plant is Cassava.

In one aspect, the hydroxynitrile lyase is from Manihot esculenta, Hevea Brasiliensis, or Baliospermum montanum. In one aspect, the hydroxynitrile lyase is from Manihot esculenta.

In one aspect, the hydroxynitrile lyase has at least 80% amino acid sequence identity to SEQ ID NO:15. In one aspect, the hydroxynitrile lyase has at least 90% amino acid sequence identity to SEQ ID NO:15. In one aspect, the hydroxynitrile lyase has at least 95% amino acid sequence identity to SEQ ID NO:15.

In one aspect, the promoter is from a potato class I patatin promoter. In one aspect, the promoter has at least 80% nucleotide sequence identity to a class I patatin promoter (SEQ ID NO:13). In one aspect, the promoter has at least 90% nucleotide sequence identity to a class I patatin promoter (SEQ ID NO:13). In one aspect, the promoter has at least 95% nucleotide sequence identity to a class I patatin promoter (SEQ ID NO:13). In one aspect, the promoter comprises SEQ ID NO:13. In one aspect, the promoter consists essentially of SEQ ID NO:13.

In another embodiment, the current invention includes a flour or starch obtainable by isolating starch from a tuber of a cassava plant,
wherein the cassava plant comprises a heterologous nucleic acid sequence comprising a promoter that is operatively linked to a nucleic acid sequence encoding hydroxynitrile lyase,
wherein the promoter drives expression of the hydroxynitrile lyase substantially exclusively in the root or tuber of the cassava plant; and
wherein the starch or flour has a protein content 50% higher than a starch or flour isolated from a wild type cassava plant using similar isolation conditions.

In one aspect, the flour has a protein content at least 75% higher than a starch isolated from a wild type cassava plant using similar isolation conditions. In one aspect, the flour has a protein content at least 100% higher than a starch isolated from a wild type cassava plant using similar isolation conditions. In one aspect, the flour has a protein content at least 200% higher than a starch isolated from a wild type cassava plant using similar isolation conditions. In one aspect, starch is extracted from the pulp of the cassava tuber.

In one aspect, the flour is isolated by a method comprising the steps of:
i) washing the tuber, followed by grating and milling it;
ii) separating starch from fibers and juice in a separator;
iii) sieving the starch;
iv) washing the starch; and
v) drying the starch.

In another embodiment the current invention includes a processed food product formed from any of the flours listed above.

### BRIEF DESCRIPTION OF DRAWINGS

A better understanding of the features and advantages of the present invention can be obtained by reference to the following detailed description that sets forth illustrative embodiments, in which the principles of the invention are utilized, and the accompanying drawings, in which:

FIG. 1: Molecular Evolutionary Genomic Analysis (MEGA4). The figure shows multiple amino acid sequence alignments of cassava HNL and rubber tree HNLs. Both HNL proteins share 77% sequence identity.

FIG. 2: Copy Number and PCR analysis of Transgenic Cell lines. Panel (A) shows a schematic graph depicting the copy numbers of the HNL transgenic lines analyzed by dot blots. Panel (B) shows a sample of PCR showing the presence of HNL and a patatin gene in the transgenic lines. Controls included water (Negative control), WT-60444, and plasmid (Positive control).

FIG. 3: Overexpression of HNL Using Patatin Promoter. Relative expression levels (Q-RT-PCR) of HNL in twelve independent transgenic lines with patatin promoter and CaMV 35S: HNL transgenic line. Tissues (both roots and leaves) were collected at 1.5 month old in vitro stage. Expression of these mRNAs relative to that of tubulin was determined. WT-60444 was adjusted to a value of 1, and all other expression values were normalized relative to this tissue. Number above the white bars (roots) indicates the fold increase compared to WT. Error bars represent SE of three biological replicates.

FIG. 4: HNL Activity Increases in Transgenic Roots. Specific activity of HNL compared with the control plants (A) roots, (B) leaves, shown by the relative amounts of hydroxynitrile lyase remaining after 1 hr incubation. Protein extracts were obtained from root and leaf tissues, and HNL enzyme activity (cyanide production) was measured colorimetrically by measuring the absorbance at 585 nm. Data are presented as relative amounts of cyanide per mg of protein after 1 hr incubation. Error bars indicate SE of the mean of three biological replicates.

FIG. 5: Immunoblots of Transgenic Cassava Expressing HNL Protein. Different amounts of protein (as indicated) from transgenic and control roots and leaves are separated by SDS-PAGE and transferred to nylon membranes. Membranes were probed first with anti-HNL antibody (1:1000) followed by secondary antibody (Anti-rabbit IgG antibody conjugated to horseradish peroxidase) at 1:10000 dilution. Detection was performed by chemiluminescence using Luminal, Iodophenol, and Hydrogen Peroxide (Sigma), followed by exposure to X-ray films for (A) 3 seconds and (B) 3 minutes.

FIG. 6: Overexpression of HNL decreases Cyanide levels in Transgenic Roots. (A). Hydrogen cyanide levels were measured using Cyanide Ion Selective Electrode in seven month old transgenic and control cassava roots. Data are presented as total amounts of cyanide per gram fresh weight of tissues. Error bars indicate SE of the mean of four biological replicates. (B). Acetone cyanohydrin levels were measured using seven month old root cortex tissues at different time intervals from 0 min to 90 minutes (half-hour intervals) post-homogenization. Data are presented as the amount of acetone cyanohydrin levels calculated as the difference between the two assays {(acetone cyanohydrin+cyanide assay)−(cyanide assay)}. Error bars indicate SE of the mean of three biological replicates. (C). GC-MS quantification of root linamarin contents in wild-type and transgenic plants. Samples were normalized with internal standard phenyl β-glucopyranoside (PGP). Linamarin content is expressed as μmoles per gram dry weight. Error bars indicate SE of the mean of four biological replicates.

FIG. 7: Overexpression of HNL Increases Protein Concentrations. Measurement of total protein concentrations of (A) roots and (B) leaves of transgenic and control plants. Total protein was extracted and measured using CB-X assay kit. Data are presented as total protein in μg/mg total dry weight of tissue. Error bars indicate SE of the mean of three biological replicates.

FIG. 8: Total and Free Amino Acid Analysis in Transgenic Cassava Roots. (A). Measurement of total hydrolyzed amino acid concentrations of transgenic and wild type roots. Samples were hydrolyzed with HCl and subjected to ACQUITY UPLC® System. Data are presented as nmoles/mg total dry weight of tissue. Error bars indicate SE of the mean of two biological replicates. (B). Measurement of free amino acid concentrations of transgenic and wild type roots. Data are presented as nmoles/mg total dry weight of tissue. Error bars indicate SE of the mean of two biological replicates.

FIG. 9: Total Amino Acids Composition of Roots of Transgenic Cassava Plants and Wild-Type Controls. Error bars represent SE for two biological replicates. Each amino acid is expressed using standard three letter abbreviations.

FIG. 10: Free Amino Acids Composition of Roots of Transgenic Cassava Plants and Wild-Type Controls. Error bars represent SE for two biological replicates. Each amino acid is expressed using standard three letter abbreviations.

FIG. 11: Predicted Amino Acid Composition of HNL Protein Using ProtParam tool (ExPasy proteomics Server). (A). Shows the classification of HNL protein into essential and non-essential amino acids. (B). Shows the individual % of amino acid composition of essential amino acids.

FIG. 12: Hypothetical Model of Overexpression of HNL using Patatin promoter in Transgenic Cassava. Proposed pathway of linamarin synthesis and breakdown in wild-type and transgenic cassava overexpressing HNL using tuber specific promoter is shown. In transgenic cassava, over-expression of HNL leads to: 1. Decrease in steady state levels of linamarin in roots, decrease in both acetone cyanohydrin and cyanide levels after processing; 2. Increase in total HNL protein, which ultimately leads to increase in total protein level in roots. Two small blue arrows indicate either decrease or increase levels in roots.

FIG. 13: Correlation Between Increasing Protein Concentrations and Decreasing Cyanide Levels in Transgenic Cassava Plants. Seven month old protein measurements are indicated as blue line in %, and cyanide detection using electrode is indicated as red line in %. Error bars represent SE for three biological replicates.

### DETAILED DESCRIPTION OF THE INVENTION

### Definitions

In order that the present disclosure may be more readily understood, certain terms are first defined. Additional definitions are set forth throughout the detailed description. As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The terms “about” or “approximately” mean within an acceptable error range for the particular value as determined by one of ordinary skill in the art, which will depend in part on how the value is measured or determined, i.e., the limitations of the measurement system. For example, “about” can mean within 1 or 2 standard deviations, from the mean value. Alternatively, “about” can mean plus or minus a range of up to 20%, preferably up to 10%, more preferably up to 5%, and more preferably up to 2.5%.

As used herein, the terms “cell,” “cells,” “cell line,” “host cell,” and “host cells,” are used interchangeably, and encompass animal cells and include plant, invertebrate, non-mammalian vertebrate, insect, algal, and mammalian cells. All such designations include cell populations and progeny. Thus, the terms “transformants” and “transfectants” include the primary subject cell and cell lines derived therefrom without regard for the number of transfers.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids can be defined where amino acids within a group exchange preferentially with each other, and therefore resemble each other most in their impact on the overall protein structure (Schulz, G. E. and R. H. Schirmer, Principles of Protein Structure, Springer-Verlag).

Examples of amino acid groups defined in this manner include: a “charged/polar group,” consisting of Glu, Asp, Asn, Gln, Lys, Arg, and His; an “aromatic, or cyclic group,” consisting of Pro, Phe, Tyr, and Trp; and an “aliphatic group” consisting of Gly, Ala, Val, Leu, Ile, Met, Ser, Thr, and Cys.

Within each group, subgroups can also be identified. For example, the group of charged/polar amino acids can be sub-divided into the sub-groups consisting of the “positively-charged sub-group,” consisting of Lys, Arg, and His; the “negatively-charged sub-group,” consisting of Glu and Asp, and the “polar sub-group” consisting of Asn and Gln. The aromatic or cyclic group can be sub-divided into the sub-groups consisting of the “nitrogen ring sub-group,” consisting of Pro, His, and Trp; and the “phenyl sub-group” consisting of Phe and Tyr. The aliphatic group can be sub-divided into the sub-groups consisting of the “large aliphatic non-polar sub-group,” consisting of Val, Leu, and Ile; the “aliphatic slightly-polar sub-group,” consisting of Met, Ser, Thr, and Cys; and the “small-residue sub-group,” consisting of Gly and Ala.

Examples of conservative mutations include substitutions of amino acids within the sub-groups above, for example Lys for Arg and vice versa, such that a positive charge can be maintained; Glu for Asp and vice versa, such that a negative charge can be maintained; Ser for Thr, such that a free —OH can be maintained; and Gln for Asn, such that a free —NH₂ can be maintained.

The term “cyanogenic glycoside” refers to any molecule in which a sugar group is bonded through its anomeric carbon to a cyanide group via a glycosidic bond. There are approximately 25 naturally occurring cyanogenic glycosides known, and common examples include Linamarin, Dhurrin, Triglochinin, and Amygdalin. The distribution of the cyanogenic glycosides in the plant kingdom is relatively wide, with at least 2500 species having some detectable cyanogenic glycoside content, with many of these species belonging to the Fabaceae, Rosaceae, Gramineae, Euphorbiaceae, Olacsceae and Linaceae families Plants of major nutritional or economic significance that produce cyanogenic glycosides include, for example, Cassava (Manihot esculenta), Sorghum (Sorghum vulgare), Flax (Linum usitatissimum), Lima beans (Phaseolus lunatus), Giant taro (Alocasia macrorrhizos), Bamboo (Bambusa arundinacea), Apple (Malus spp.), Peach (Prunus persica), Nectarine (Prunus persica var nucipersica), Cherry (Prunus spp.), Bitter almond (Prunus dulcis), raspberry, and crabapple.

Cyanogenic glycoside content is usually reported in terms of the total HCN content of the fresh tissue in mg HCN/kg. Cyanogen levels can vary widely with cultivar, climatic conditions, plant part, and degree of processing. Typical levels for some plant materials consumed by humans are presented in Table D1:

TABLE D1

| | Major | |
|---|---|---|
| | cyanogenic | Cyanogen |
| | glycoside | content |
| Food | present | (mg HCN/kg) |
| Cassava (Manihot esculenta) - root | Linamarin | 15-1000 |
| Sorghum (Sorghum vulgare) - leaves | Dhurrin | 750-790 |
| Flax (Linum usitatissimum) - seed meal | Linamarin, | 360-390 |
| | linustatin, | |
| | neolinustatin | |
| Lima beans (Phaseolus lunatus) | Lotaustralin | 2000-3000 |
| Giant taro (Alocasia macrorrhizos) - | Triglochinin | 29-32 |
| leaves | | |
| Bamboo (Bambusa arundinacea) - | Taxiphyllin | 100-8000 |
| youngshoots | | |
| Apple (Malus spp.) - Seed | Amygdalin | 690-790 |
| Peach (Prunus persica) - Kernel | Amygdalin | 710-720 |
| Apricot (Prunus armeniace) - Kernel | Amygdalin | 89-2170 |
| | | 2.2 (juice) |
| Plum (Prunus spp.) - Kernel | Amygdalin | 696-764 |
| Nectarine | Amygdalin | 196-209 |
| (Prunus persica var nucipersica) - Kernel | | |
| Cherry (Prunus spp.) | Amygdalin | 4.6 (juice) |
| Bitter almond (Prunus dulcis) | Amygdalin | 4700 |

Accordingly, the term “plant that produces cyanogenic glucosides” refers to a plant that has a cyanogen content of greater than about 30 mg HCN/kg of fresh tissue, in any part of the plant.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence. For example, mRNA transcribed from a selected sequence can be quantified by Northern blot hybridization, ribonuclease RNA protection, in situ hybridization to cellular RNA, or by PCR. Proteins encoded by a selected sequence can be quantified by various methods including, but not limited to, e.g., ELISA, Western blotting, radioimmunoassays, immunoprecipitation, assaying for the biological activity of the protein, or by immunostaining of the protein followed by FACS analysis.

“Expression control sequences” are regulatory sequences of nucleic acids, such as promoters, leaders, enhancers, introns, recognition motifs for RNA or DNA binding proteins, polyadenylation signals, terminators, internal ribosome entry sites (IRES), and the like, that have the ability to affect the transcription or translation of a coding sequence in a host cell. Exemplary expression control sequences are described in Goeddel; Gene Expression Technology: Methods in Enzymology 185, Academic Press, San Diego, Calif. (1990).

A “gene” is a sequence of nucleotides that codes for a functional gene product. Generally, a gene product is a functional protein. However, a gene product can also be another type of molecule in a cell, such as RNA (e.g., a tRNA or an rRNA). A gene may also comprise regulatory (i.e., non-coding) sequences, as well as coding sequences and introns. Exemplary regulatory sequences include promoters, enhancers, and terminators. The transcribed region of the gene may also include untranslated regions including introns, a 5′-untranslated region (5′-UTR) and a 3′-untranslated region (3′-UTR).

The term “heterologous” refers to nucleic acids or proteins that have been introduced into a plant, or animal, or cell, or a nucleic acid molecule (such as a chromosome, vector, or nucleic acid construct), that is derived from another source, or which is from the same source but which is located in a different (i.e., non-native) context or location.

The term “homology” describes a mathematically-based comparison of sequence similarities that is used to identify genes or proteins with similar functions or motifs. The nucleic acid and protein sequences of the present invention can be used as a “query sequence” to perform a search against public databases to, for example, identify other family members, related sequences, or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12, to obtain nucleotide sequences homologous to nucleic acid molecules of the invention. BLAST protein searches can be performed with the XBLAST program, score=50, wordlength=3, to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used.

The term “homologous” refers to the relationship between two proteins that possess a “common evolutionary origin”, including proteins from superfamilies (e.g., the immunoglobulin superfamily) in the same species of animal, as well as homologous proteins from different species of animals (for example, myosin light chain polypeptide, etc.; see Reeck et al., Cell, 50:667, 1987). Such proteins (and their encoding nucleic acids) have sequence homology, as reflected by their sequence similarity, whether in terms of percent identity or by the presence of specific residues or motifs, and conserved positions.

As used herein, the term “increase”, or the related terms “increased”, “enhance”, or “enhanced”, refers to a statistically significant increase. For the avoidance of doubt, the terms generally refer to at least a 10% increase in a given parameter, and can encompass at least a 20% increase, 30% increase, 40% increase, 50% increase, 60% increase, 70% increase, 80% increase, 90% increase, 95% increase, 97% increase, 99% increase, or even a 100% increase, over the control value.

The term “isolated,” when used to describe a protein or nucleic acid, means that the material has been identified and separated and/or recovered from a component of its natural environment. Contaminant components of its natural environment are materials that would typically interfere with research, diagnostic, or therapeutic uses for the protein or nucleic acid, and may include enzymes, hormones, and other proteinaceous or non-proteinaceous solutes. In some embodiments, the protein or nucleic acid will be purified to at least 95% homogeneity as assessed by SDS-PAGE under non-reducing or reducing conditions using Coomassie blue or, preferably, silver stain. Isolated protein includes protein in situ within recombinant cells, since at least one component of the protein of interest's natural environment will not be present. Ordinarily, however, isolated proteins and nucleic acids will be prepared by at least one purification step.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs.

Optimal alignment of sequences for comparison can be conducted, for example, by the local homology algorithm described in Smith & Waterman 1981, by the homology alignment algorithm described in Needleman & Wunsch 1970, by the search for similarity method described in Pearson & Lipman 1988, by computerized implementations of these algorithms (GAP, BESTFIT, PASTA, and TFASTA in the GCG Wisconsin Package, available from Accelrys, Inc., San Diego, Calif., United States of America), or by visual inspection. See generally, (Altschul, S. F. et al., J. Molec. Biol. 215: 403-410 (1990) and Altschul et al. Nucl. Acids Res. 25: 3389-3402 (1997)).

One example of an algorithm that is suitable for determining percent sequence identity and sequence similarity is the BLAST algorithm, which is described in Altschul, S., et al., NCBI NLM NIH Bethesda, Md. 20894; and Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold.

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always; 0) and N (penalty score for mismatching residues; always; 0). For amino acid sequences, a scoring matrix is used to calculate the cumulative score. Extensions of the word hits in each direction are halted when the −27 cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

The terms “operably linked” and “operatively linked,” as used interchangeably herein, refer to the positioning of two or more nucleotide sequences or sequence elements in a manner which permits them to function in their intended manner. In some embodiments, a nucleic acid molecule according to the invention includes one or more DNA elements capable of opening chromatin and/or maintaining chromatin in an open state operably linked to a nucleotide sequence encoding a recombinant protein. In other illustrative embodiments, a nucleic acid molecule may additionally include one or more DNA or RNA nucleotide sequences chosen from: (a) a nucleotide sequence capable of increasing translation; (b) a nucleotide sequence capable of increasing secretion of the recombinant protein outside a cell; (c) a nucleotide sequence capable of increasing the mRNA stability, and (d) a nucleotide sequence capable of binding a trans-acting factor to modulate transcription or translation, where such nucleotide sequences are operatively linked to a nucleotide sequence encoding a recombinant protein. Generally, but not necessarily, the nucleotide sequences that are operably linked are contiguous and, where necessary, in reading frame. However, although an operably linked DNA element capable of opening chromatin and/or maintaining chromatin in an open state is generally located upstream of a nucleotide sequence encoding a recombinant protein, it is not necessarily contiguous with it. Operable linking of various nucleotide sequences is accomplished by recombinant methods well known in the art, e.g., using PCR methodology, by ligation at suitable restrictions sites, or by annealing. Synthetic oligonucleotide linkers or adaptors can be used in accord with conventional practice if suitable restriction sites are not present.

The terms “polynucleotide,” “nucleotide sequence,” and “nucleic acid” are used interchangeably herein, and refer to a polymeric form of nucleotides of any length, either ribonucleotides or deoxyribonucleotides, and can consist of, consist essentially of, or comprise the particular sequences indicated herein. These terms include a single-, double-, or triple-stranded DNA, genomic DNA, cDNA, RNA, DNA-RNA hybrid, or a polymer comprising purine and pyrimidine bases, or other natural, chemically or biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. A nucleic acid molecule can take many different forms, e.g., a gene or gene fragment, one or more exons, one or more introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracil, other sugars and linking groups such as fluororibose and thioate, and nucleotide branches. As used herein, a polynucleotide includes not only naturally occurring bases such as A, T, U, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Prokaryotic promoters contain Shine-Dalgarno sequences in addition to the −10 and −35 consensus sequences.

A large number of promoters, including constitutive, inducible, and repressible promoters, from a variety of different sources are well known in the art. Representative sources include for example, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters active in plants include, for example the nopaline synthase (nos) promoter and octopine synthase (ocs) promoter carried on tumor-inducing plasmids of Agrobacterium tumefaciens, and the caulimovirus promoters such as the Cauliflower Mosaic Virus (CaMV) 19S or 35S promoter (U.S. Pat. No. 5,352,605), CaMV 35S promoter with a duplicated enhancer (U.S. Pat. Nos. 5,164,316; 5,196,525; 5,322,938; 5,359,142; and 5,424,200), the Figwort Mosaic Virus (FMV) 35S promoter (U.S. Pat. No. 5,378,619), and the cassava vein mosaic virus (U.S. Pat. No. 7,601,885). These promoters and numerous others have been used in the creation of constructs for transgene expression in plants or plant cells. Other useful promoters are described, for example, in U.S. Pat. Nos. 5,391,725; 5,428,147; 5,447,858; 5,608,144; 5,614,399; 5,633,441; 6,232,526; and 5,633,435, all of which are incorporated herein by reference.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell. Methods for purification are well-known in the art. As used herein, the term “substantially free” is used operationally, in the context of analytical testing of the material. Preferably, purified material substantially free of contaminants is at least 50% pure; more preferably, at least 75% pure, and more preferably still at least 95% pure. Purity can be evaluated by chromatography, gel electrophoresis, immunoassay, composition analysis, biological assay, and other methods known in the art. The term “substantially pure” indicates the highest degree of purity that can be achieved using conventional purification techniques known in the art.

The term “sequence similarity” refers to the degree of identity or correspondence between nucleic acid or amino acid sequences that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In specific embodiments, two nucleic acid sequences are “substantially homologous” or “substantially similar” when at least about 85%, and more preferably at least about 90%, or at least about 95%, of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

The term “specific” in the context of “specific binding” is applicable to a situation in which one member of a specific binding pair will not show any significant binding to molecules other than its specific binding partner(s). The term is applicable, for example, to the situation where two complementary polynucleotide strands can anneal together, yet each single stranded polynucleotide exhibits little or no binding to other polynucleotide sequences under stringent hybridization conditions.

The term “storage protein” refers to a protein that is typically present in the storage organ in a plant at an amount that is greater than about five percent, or a protein with hydroxynitrile lyase activity. Examples of storage proteins include for example, arachins, avenins, cocosins, conarchins, concocosins, conglutins, conglycinins, convicines, crambins, cruciferins, cucurbitins, dioscorins, edestins, excelesins, gliadins, glutens, glytenins, glycinins, helianthins, hordeins, kafirins, legumins, napins, oryzins, pennisetins, phaseolins, prolamines, psophocarpins, secalins, sporamins, tryspsin inhibitors, vicilins, vicines, zeins, and hydroxynitrile lyase.

In particular embodiments of the invention, two amino acid sequences are “substantially homologous” or “substantially similar” when greater than 90% of the amino acid residues are identical. Two sequences are functionally identical when greater than about 95% of the amino acid residues are similar. Preferably, the similar or homologous polypeptide sequences are identified by alignment using, for example, the GCG (Genetics Computer Group, Version 7, Madison, Wis.) pileup program, or using any of the programs and algorithms described above. The program may use the local homology algorithm of Smith and Waterman with the default values: Gap creation penalty=−(1+1/k), k being the gap extension number, Average match=1, Average mismatch=−0.333.

As used herein, a “transgenic plant” is one whose genome has been altered by the incorporation of heterologous genetic material, e.g., by transformation as described herein. The term “transgenic plant” is used to refer to the plant produced from an original transformation event, or progeny from later generations or crosses of a transgenic plant, so long as the progeny contains the heterologous genetic material in its genome.

The term “transformation” or “transfection” refers to the transfer of one or more nucleic acid molecules into a host cell or organism. Methods of introducing nucleic acid molecules into host cells include, for example, calcium phosphate transfection, DEAE-dextran mediated transfection, microinjection, cationic lipid-mediated transfection, electroporation, scrape loading, ballistic introduction, Agrobacterium infection, or infection with viruses or other infectious agents.

“Transformed”, “transduced”, or “transgenic”, in the context of a cell, refers to a host cell or organism into which a recombinant or heterologous nucleic acid molecule (e.g., one or more DNA constructs or RNA, or siRNA counterparts) has been introduced. The nucleic acid molecule can be stably expressed (i.e., maintained in a functional form in the cell for longer than about three months) or non-stably maintained in a functional form in the cell for less than three months, i.e., is transiently expressed. For example, “transformed,” “transformant,” and “transgenic” cells have been through the transformation process and contain foreign nucleic acid. The term “untransformed” refers to cells that have not been through the transformation process.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Buchanan et al., Biochemistry and Molecular Biology of Plants, Courier Companies, USA, 2000; Mild and Iyer, Plant Metabolism, 2^{nd} Ed. D. T. Dennis, D H Turpin, D D Lefebrve, D G Layzell (eds) Addison Wesly, Langgmans Ltd. London (1997); and Lab Ref: A Handbook of Recipes, Reagents, and Other Reference Tools for Use at the Bench, Edited by Jane Roskams and Linda Rodgers, 2002, Cold Spring Harbor Laboratory, ISBN 0-87969-630-3. Each of these general texts is herein incorporated by reference.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

The publications discussed above are provided solely for their disclosure before the filing date of the present application. Nothing herein is to be construed as an admission that the invention is not entitled to antedate such disclosure by virtue of prior invention.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### I. Overview of Methods

The present invention includes methods and transgenic plants that exhibit reduced levels of cyanogenic glycosides and improved protein content. In one aspect, such methods and transgenic plants are created through the tissue-specific expression of a storage protein, and/or hydroxynitrile lyase (HNL) in the roots and tubers of the plants.

In one aspect of any of these methods, the storage protein is operatively coupled to targeting sequences that target secretion of the storage protein to the apoplastic space. In one aspect, such targeting is achieved via the use of targeting sequences from a native HNL or linamarase.

Accordingly, in one aspect, the present invention includes a method of increasing the protein content of plant that produces cyanogenic glucosides, said method comprising:
i) transforming the plant with at least one nucleic acid molecule comprising a promoter preferentially active in a root or storage organ operatively linked to at least one transgene that encodes a plant hydroxynitrile lyase, to produce a transformed plant;
ii) selecting the transformed plant comprising the at least one transgene;
iii) growing the transformed plant to produce a plant exhibiting increased protein content when compared to an equivalent non-transformed plant, wherein the transformed plant and the equivalent non-transformed plant are grown under similar conditions.

In another embodiment, the current invention includes a method of preferentially decreasing the cyanide content in the roots and tuber storage cells of a plant that produces cyanogenic glucosides, said method comprising:
i) transforming the plant with at least one nucleic acid molecule comprising a promoter preferentially active in a root or storage organ operatively linked to at least one transgene that encodes a plant hydroxynitrile lyase, to produce a transformed plant;
ii) selecting the transformed plant comprising the at least one transgene;
iii) growing the transformed plant to produce a plant exhibiting decreased cyanide content in the root and tuber storage cells, but not the leaves, when compared to an equivalent non-transformed plant, wherein the transformed plant and the equivalent non-transformed plant are grown under similar conditions; and
   wherein the promoter drives expression of the hydroxynitrile lyase substantially exclusively in the root or tuber storage cells of the transgenic plant.

In another embodiment, the current invention includes a method for producing hydroxynitrile lyase from a transgenic plant that produces cyanogenic glucosides, comprising:
i) cultivating the transgenic plant, wherein the transgenic plant comprises a heterologous nucleic acid sequence comprising a promoter that is operatively linked to a nucleic acid sequence encoding hydroxynitrile lyase,
   wherein the promoter drives expression of the hydroxynitrile lyase substantially exclusively in the root or tuber storage cells of the cassava plant; and
ii) isolating the hydroxynitrile lyase from the tubers of the cultivated plants.

### II. Promoters

The expression of the storage protein and/or hydroxynitrile lyases (HNL) in any of the plants and methods of the invention is controlled by expression control sequences, including promoters and 5′ and 3′ flanking sequences that provide for transcription in a tissue-specific manner within the root or tuber cells. Specificity in this context means that a promoter is mainly or exclusively active in the root, or tuber storage cells of a plant. Accordingly, a promoter that is preferentially active in a root or storage organ (i.e., is root-specific) for example shows expression in roots or tuber storage cells at detectable levels (as measured by, for example, RNA blots) which are, under comparable experimental conditions, detectable in above-ground organs of the plant (i.e., stem, petioles, leaves, and blossoms) at less than 30%, preferably less than 20%, and more preferably less than 15%, of the level in roots or tuber storage cells. This specificity is not restricted to a particular experimental time point, but is generally present during the entire vegetation period.

In one aspect of any of these methods, and plants, the promoter drives expression of the storage protein and/or hydroxynitrile lyase substantially exclusively in the root or tuber of the plant, meaning that the expression from the promoter in non-root tissues is less than about 10% of that observed in the root or tuber storage cells, at any given time point.

In one aspect of any of these methods, and plants, the storage protein comprises targeting sequences that direct secretion of the storage protein to the apoplastic space. In one aspect, such targeting is achieved via the use of signal sequences from a native cassava HNL (MVTAHFVLIHTICHG (SEQ ID NO:18)) or linamarase (MLVLFISLLALTRPAMG (SEQ ID NO:19).

Many root-specific promoters have been identified in cassava and other plants including, for example, the class I patatin promoter (Ihemere et al., (2006) Plant Biotechnol. J. 4: 453-465), as well as promoters disclosed, for example, in Arango et al., (2010) Putative storage root-specific promoters from cassava and yam: cloning and evaluation in transgenic carrots as a model system. Plant Cell Rep.; 29(6):651-9; de Souza C R et al., (2009) Isolation and characterization of the promoter sequence of a cassava gene coding for Pt2L4, a glutamic acid-rich protein differentially expressed in storage roots. Genet Mol Res. 8(1):334-44; Oltmanns et al., (2006) Taproot promoters cause tissue specific gene expression within the storage root of sugar beet. Planta 224(3):485-95; Xiao et al., (2006) Isolation and characterization of root-specific phosphate transporter promoters from Medicago trunatula. Plant Biol. (Stuttg) 8(4):439-49; and Zhang et al., (2003) Two cassava promoters related to vascular expression and storage root formation. Planta 218(2):192-203.

Numerous additional root-specific promoters have been described for various plant species in the patent literature. Representative promoters include, for example, those described in U.S. Pat. Nos. U.S. Pat. No. 5,436,393; U.S. Pat. No. 5,459,252; U.S. Pat. No. 5,723,757; U.S. Pat. No. 5,750,399; U.S. Pat. No. 5,837,876; U.S. Pat. No. 5,959,176; U.S. Pat. No. 7,767,801; and published US and PCT Application Nos. US20040031074A1; US20050010974A1; US20080244791A; US20100269225A1; and WO2009104893A1.

Accordingly, suitable root-specific promoters from these sources are readily available, or can be made synthetically, based on sequences publicly available on line or, for example, from depositories of plants, as well as various commercial or individual sources.

In one aspect of the invention, a root-specific promoter comprises a nucleotide sequence derived from a class I patatin promoter from potato. In one aspect, the nucleotide sequence may comprise about 200 nucleotides from the patatin promoter, in another aspect, about 400 nucleotides, in another aspect, about 600 nucleotides, in another aspect about 800 nucleotides, and in another aspect, about 1000 nucleotides or more.

Patatin is a family of glycoproteins that possess lipid acyl hydrolase and transferase activities (Andrews et al., (1988) Biochem. J. 252: 199-206) and account for up to 40% of the total soluble protein in potato tubers (Paiva et al., (1983) Plant Physiol. 71: 161-168). Genes encoding patatin, the major storage protein of the potato tuber, are generally divided into two classes, class I and class II. The expression of the class I patatin genes is normally tuber-specific, but can be induced in leaves by high concentrations of sucrose (Kim et al., (1994) Plant Mol. Biol. 26: 603-615), whereas patatins detected in roots and other epidermal cells are primarily derived from class-II genes (Pikaard et al., (1987) Nucleic Acids Res. 15: 1979-1994; Mignery et al., (1988) Gene 62: 27-44), which are found to be not sucrose-inducible (Köster-Töpfer et al., (1989) Mol. Gen. Genet. 219: 390-396; Liu et al., (1990) Mol. Gen. Genet. 223: 401-406). Storage-root-specificity in cassava (Ihemere et al., (2006) Plant Biotechnol. J. 4: 453-465) and root specificity in A. thaliana (Martin et al., (1997) Plant J. 11:53-62) have been previously documented, and class I patatin promoters are widely used by the cassava research community.

The sequence of a representative Class I patatin promoter is provided in SEQ ID NO:13. Also included in the term “class I patatin promoter” are nucleotide sequences that are shortened or elongated but otherwise identical versions of SEQ ID NO:13, as well as homologs and derivatives of the class I patatin promoter from other species or varieties with the same, or similar, expression characteristics, as well as chimeric promoters comprising one or more elements of the patatin promoter coupled to other expression control sequences.

The term “derivative” includes a nucleotide sequence that may have a different overall nucleotide sequence, but which has a similar functional characteristic compared to the nucleotide sequence of SEQ ID NO:13. Specifically, the above-described promoter sequence and 5′-UTR sequence may have a nucleotide sequence that has a sequence identity of at least 70%, preferably at least 80%, more preferably at least 85%, more preferably at least 90%, still more preferably at least 95%, and still more preferably at least 98%, compared to the nucleotide sequence of SEQ ID NO:13.

### III. Hydroxynitrile Lyase Genes

Hydroxynitrile lyases (HNLs) catalyze the cleavage of cyanohydrins to yield hydrocyanic acid plus the corresponding aldehyde or ketone. In the presence of high concentrations of HCN and aldehydes or ketones, HNLs can be used as biocatalysts for the stereoselective synthesis of enantiomerically pure cyanohydrins, several of which are important building blocks in the pharmaceuticals and fine chemical industries (Purkarthofer et al., (2007) Appl Microbiol Biotechnol. 76(2):309-20). For example, acetone cyanohydrin is used in the production of methyl methacrylate, the monomer of the transparent plastic polymethyl methacrylate (PMMA), also known as acrylic.

Several different classes of HNLs are known to date, some of which contain an FAD cofactor (FAD-HNL). Despite catalyzing the same reaction, they differ in substrate specificity, and do not share any significant homology on either the sequence or structural level. Due to these substantial differences, HNLs from different species are believed to have evolved from unrelated precursor proteins by convergent evolution.

To date, 3D structures are known for four HNLs. Three of them, the HNLs from Hevea brasiliensis (HbHNL) (Wagner et al., (1996) Structure. 4(7):811-22), Manihot esculenta (Lauble et al., (2001) Protein Sci. 10(5):1015-22), and Sorghum bicolor (SbHNL), adopt α/β-hydrolase folds. The fourth, the FAD-HNL from Prunus amygdalus (almond, PaHNL), closely resembles glucose-methanol-choline (GMC) oxidoreductases despite not catalyzing a redox reaction.

Hydroxynitrile lyases from cassava, Manihot esculenta (MeHNL), and Hevea brasiliensis (HbHNL) catalyze the formation of (S)-cyanohydrins from HCN and aldehydes or ketones, and belong to the α/β-hydrolase superfamily. All α/β hydrolase fold enzymes have a “nucleophile-histidine-acid” catalytic triad found in common with the subtilisin and chymotrypsin class of serine proteases. In all these enzymes, the nucleophile is part of the consensus motif Gly-X-Ser/Cys-X-Gly/Ala-Gly/Ala (SEQ ID NO: 14) (Ollis et al., (1992) Protein Eng. 5:197-211).

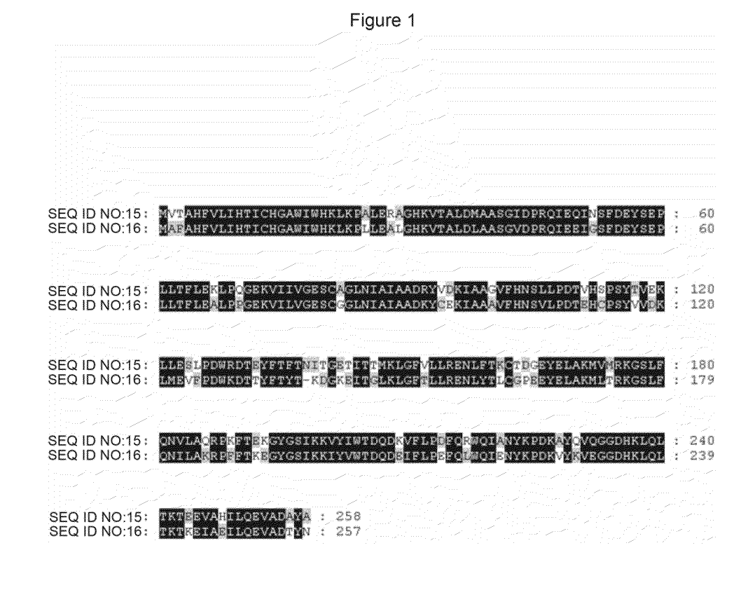

There is functional evidence by site-directed mutagenesis for the use of a catalytic triad by MeHNL and HbHNL as well (Hasslacher et al., (1996) J. Biol. Chem. 271, 5884-5891; Wajant and Pfizenmaier (1996) J. Biol. Chem. 271, 25830-25834). Moreover, the order of the catalytic triad residues in the primary sequence suggests that these HNLs also belong to the α/β hydrolase fold group of enzymes despite having no sequence homologies to SbHNL. The hydroxynitrile lyase from cassava, MeHNL, has 77% sequence identity with the deduced amino acid sequence of the rubber tree hydroxynitrile lyase (Hevea brasiliensis) (HbHNL) (FIG. 1).

The terms “hydroxynitrile lyase” or “HNL” refer to enzymes capable of the hydrolysis of acetone cyanohydrin to cyanide and acetone. Exemplary genes encoding HNL include those listed in Table D2. In one aspect, the hydroxynitrile lyase is from a plant that produces a cyanogenic glycoside. In a further embodiment, the hydroxynitrile lyase is selected from the group consisting of Manihot esculenta HNL, Hevea Brasiliensis HNL, and Baliospermum montanum HNL. In another aspect, the hydroxynitrile lyase is from Manihot esculenta. Representative species and GenBank accession numbers for various species of plant that are potential sources of HNL are listed below in Table D2, and genes from other species can be readily identified by standard homology searching of publicly available databases based on the conserved catalytic cleft motif (SEQ ID NO:14) of the HNLs.

TABLE D2

| Species and | | |
|---|---|---|
| Accession | | |
| number | Sequence | SEQ.ID.NO. |
| Manihot | MVTAHFVLIH TICHGAWIWH KLKPALERAG HKVTALDMAA | SEQ.ID.NO. 15 |
| esculenta | SGIDPRQIEQ INSFDEYSEP LLTFLEKLPQ GEKVIIVGES | |
| AAV52632.1 | CAGLNIAIAA DRYVDKIAAG VFHNSLLPDT VHSPSYTVEK | |
| | LLESLPDWRD TEYFTFTNIT GETITTMKLG FVLLRENLFT | |
| | KCTDGEYELA KMVMRKGSLF QNVLAQRPKF TEKGYGSIKK | |
| | VYIWTDQDKV FLPDFQRWQI ANYKPDKAYQ VQGGDHKLQL | |
| | TKTEEVAHIL QEVADAYA | |
| Hevea | MAFAHFVLIHT ICHGAWIWHK LKPLLEALGH KVTALDLAAS | SEQ.ID.NO. 16 |
| Brasiliensis | GVDPRQIEEI GSFDEYSEPL LTFLEALPPG EKVILVGESC | |
| 1YB6_A | GGLNIAIAAD KYCEKIAAAV FHNSVLPDTE HCPSYVVDKL | |
| | MEVFPDWKDT TYFTYTKDGK EITGLKLGFT LLRENLYTLC | |
| | GPEEYELAKM LTRKGSLFQN ILAKRPFFTK EGYGSIKKIY | |
| | VWTDQDEIFL PEFQLWQIEN YKPDKVYKVE GGDHKLQLTK | |
| | TKEIAEILQE VADTYN | |
| Baliospermum | MVSAHFILIH TICHGAWLWY KLIPLLQSAG HNATAIDLVA | SEQ.ID.NO. 17 |
| montanum | SGIDPRQLEQ IGTWEQYSEP LFTLIESIPE GKKVILVGEA | |
| BAI50634.1 | GGGINIALAA EKYPEKVSAL VFHNALMPDI DHSPAFVYKK | |
| | FSEVFTDWKD SIFSNYTYGN DTVTAVELGD RTLAENIFSN | |
| | SPIEDVELAK HLVRKGSFFE QDLDTLPNFT SEGYGSIRRV | |
| | YVYGEEDQIF SRDFQLWQIN NYKPDKVYCV PSADHKIQIS | |
| | KVNELAQILQ EVANSASDLL AVA | |

The hydroxynitrile lyase may be in its native form, i.e., as different apo forms, or allelic variants as they appear in nature, which may differ in their amino acid sequence, for example, by proteolytic processing, including by truncation (e.g., from the N- or C-terminus or both) or other amino acid deletions, additions, insertions, or substitutions.

Naturally-occurring chemical modifications including post-translational modifications and degradation products of the hydroxynitrile lyase are also specifically included in any of the methods of the invention including, for example, pyroglutamyl, iso-aspartyl, proteolytic, phosphorylated, glycosylated, reduced, oxidatized, isomerized, and deaminated variants of the hydroxynitrile lyase.

The hydroxynitrile lyase which may be used in any of the methods and plants of the present invention may have amino acid sequences that are substantially homologous, or substantially similar to, any of the native hydroxynitrile lyase amino acid sequences, for example, to any of the native hydroxynitrile lyase gene sequences listed in Table D2.

Alternatively, the hydroxynitrile lyase may have an amino acid sequence having at least 30% identity, preferably at least 40, 50, 60, 70, 75, 80, 85, 90, 95, 98, or 99% identity, with hydroxynitrile lyases listed in Table D2. In a preferred embodiment, the hydroxynitrile lyase for use in any of the methods and plants of the present invention is at least 80% identical to the mature hydroxynitrile lyase from Manihot esculenta (SEQ ID NO:15).

It is known in the art to synthetically modify the sequences of proteins or peptides, while retaining their useful activity, and this may be achieved using techniques that are standard in the art and widely described in the literature, e.g., random or site-directed mutagenesis, cleavage, and ligation of nucleic acids, or via the chemical synthesis or modification of amino acids or polypeptide chains. For instance, conservative amino acid mutations changes can be introduced into hydroxynitrile lyases, and are considered within the scope of the invention. Mutations of hydroxynitrile lyase that increase the activity of the protein are known, and may be used in the methods and plants of the invention. (See, e.g., US Patent Publication 2009/0170156). Such useful mutations include, for example, any combination of the following: i) the mutation of the second amino of any of the HNL's listed in Table D2 to an amino acid selected from the group consisting of Lys, Asn, Ile, Arg, Gln, Pro, Thr, Tyr, Leu, Met, Ser, and Glu; ii) The mutation of His 103 in any of the HNL's listed in Table D2 to an amino acid selected from the group consisting of Val, Ile, Arg, Gln, Trp, Thr, Cys, Leu, Met, Ser, and Ala; iii) The mutation of Lys 175, Lys 198, or Lys 223 of any of the HNLs listed in Table D2 by another amino acid.

The hydroxynitrile lyase may thus include one or more amino acid deletions, additions, insertions, and/or substitutions based on any of the naturally-occurring isoforms of hydroxynitrile lyase. These may be contiguous or non-contiguous. Representative variants may include those having 1 to 8, or more preferably 1 to 4, 1 to 3, or 1 or 2 amino acid substitutions, insertions, and/or deletions as compared to any of sequences listed in Table D2.

The variants, derivatives, and fusion proteins of hydroxynitrile lyase are functionally equivalent in that they have detectable hydroxynitrile lyase activity. More particularly, they exhibit at least 5%, at least 10%, at least 20%, at least 30%, at least 40%, preferably at least 60%, more preferably at least 80%, more preferably at least 90%, more preferably at least 95%, and even more preferably at least 98%, of the activity of hydroxynitrile lyase from cassava (Manihot esculenta) SEQ ID NO:15, and are thus capable of substituting for hydroxynitrile lyase itself.

Such activity means any activity exhibited by a native hydroxynitrile lyase, whether a physiological response exhibited in an in vivo or in vitro test system, or any biological activity or reaction mediated by a native hydroxynitrile lyase, e.g., in an enzyme, or cell based assay. All such variants, derivatives, fusion proteins, or fragments of the hydroxynitrile lyase are included, and may be used in any of the polynucleotides, vectors, host cells, and methods disclosed and/or claimed herein, and are subsumed under the term “hydroxynitrile lyase”.

Suitable assays for determining functional hydroxynitrile lyase activity include, for example, those disclosed in Asano et al., (2005) Screening for new hydroxynitrilases from plants. Biosci. Biotechnol. Biochem. 69(12):2349-57; and Andexer et al., (2006) A high-throughput screening assay for hydroxynitrile lyase activity. Chem. Commun. (Camb.) (40):4201-3.

### IV. Expression Vectors

A vector of this invention is a nucleic acid molecule that comprises a promoter nucleotide sequence that is preferentially active in a root or storage organ that is operatively linked to a heterologous nucleic acid sequence comprising a sequence encoding a storage protein and/or a HNL. Typically, the vector is capable of expressing the operatively linked promoter and heterologous nucleic acid sequences as a chimeric gene. Vectors suitable for use in expressing chimeric genes are generally well known, and need not be limited. A chimeric gene for use in a vector herein is a fusion between a promoter nucleotide sequence of this invention operatively linked to a heterologous nucleic acid sequence.

Expression cassettes containing any of the promoters preferentially active in a root or storage organ and any of the storage proteins and/or HNLs disclosed herein can be constructed in a variety of ways. These techniques are known to those of skill in the art, and are described generally in Sambrook, et al., Molecular Cloning—A Laboratory Manual (2nd Ed.), Vol. 1-3, Cold Spring Harbor Laboratory, Cold Spring Harbor, N.Y., 1989, which is incorporated herein by reference. For instance, various procedures, such as PCR, or site-directed mutagenesis, can be used to introduce a restriction site at the start codon of a heterologous gene fragment. Heterologous DNA sequences are then operably linked to the root-specific promoter such that the expression of the heterologous DNA sequences, encoding the HNL, are regulated by the promoter.

DNA constructs composed of a root-specific promoter operably linked to heterologous DNA sequences encoding the storage protein and/or HNL can then be inserted into a variety of vectors. Such vectors include expression vectors that are useful in the transformation of plant cells. Many other such vectors useful in the transformation of plant cells can be constructed by the use of recombinant DNA techniques well known to those of skill in the art.

Exemplary vectors for expression in protoplasts or plant tissues include pUC 18/19 or pUC 118/119 (GIBCO BRL, Inc., MD); pBluescript SK (+/−) and pBluescript KS (+/−) (STRATAGENE, La Jolla, Calif.); pT7Blue T-vector (NOVAGEN, Inc., WI); pGEM-3Z/4Z (PROMEGA Inc., Madison, Wis.), and the like vectors, such as is described herein.

Exemplary vectors for expression using Agrobacterium tumefaciens-mediated plant transformation include pBin 19 (CLONETECH), Frisch et al., Plant Mol. Biol., 27:405-409, 1995; pCAMBIA 1200 and pCAMBIA 1201 (Center for the Application of Molecular Biology to International Agriculture, Canberra, Australia); pGA482, An et al, EMBO J., 4:277-284, 1985; pCGN1547, (CALGENE Inc.) McBride et al., Plant Mol. Biol., 14:269-276, 1990, and the like vectors, such as are described herein.

Techniques for nucleic acid manipulation of genes such as subcloning a subject promoter or nucleic acid sequences encoding HNL into expression vectors, labeling probes, DNA hybridization, and the like, are described generally in Sambrook, et al., supra.

### V. Transgenic Plants

The present invention also contemplates a transgenic plant comprising a promoter preferentially active in a root or storage organ operatively linked to a nucleic acid sequence encoding the storage protein and/or HNL as described herein. The transgenic plant therefore contains an expression cassette as defined herein as a part of the plant, the cassette having been introduced by transformation of a plant with a vector of this invention. In one aspect, such transgenic plants exhibit preferentially decreased levels of cyanogenic glycosides in their roots and tuber storage cells, but essentially unchanged levels of cyanogenic glycosides in their leaves, and other non-root organs of the plant.

In one aspect, such transgenic plants are characterized by having a protein content (measured after about seven months of growth) in their leaves which is at least about 50% higher, at least about 60% higher, at least about 80% higher, at least about 100% higher, at least about 200% higher, or at least about 300% higher, than that of corresponding wild-type transgenic plants. In one aspect, such transgenic plants are characterized by a protein content in their leaves of approximately 300 to 400 μg/mg dry weight.

In one aspect, such transgenic plants are further characterized as having essentially the same leaf content of cyanogenic glycosides, as that in wild-type transgenic plants. In one aspect, the linamarin content of the leaves of the transgenic plant (measured after about seven months of growth) is approximately the same as that in wild-type transgenic plants.

In one aspect, such transgenic plants are characterized by having a protein content in their root or tuber storage cells (measured after about seven months of growth) that is at least about 20% higher, at least about 40% higher, at least about 60% higher, at least about 80% higher, at least about 100% higher, at least about 200% higher, at least about 300% higher, at least about 400% higher, or at least about 500% higher, than that of corresponding wild-type transgenic plants. In one aspect, such transgenic plants are characterized by a protein content in their roots or tuber storage cells of approximately 30 to 70 μg/mg dry weight.

In one aspect, such transgenic plants are characterized by having a total amino acid content in their root or tuber storage cells (measured after about seven months of growth) which is at least about 20% higher, at least about 40% higher, at least about 60% higher, at least about 80% higher, at least about 100% higher, at least about 150% higher, at least about 200% higher, or at least about 300% higher, than that of corresponding wild-type transgenic plants. In one aspect, such transgenic plants are characterized by a specific increase in the abundance of Gly, Asp, Glu, or Arg.

In one aspect, such transgenic plants are characterized by having a free amino acid content in their root or tuber storage cells (measured after about seven months of growth) which is at least about 20% higher, at least about 40% higher, at least about 60% higher, at least about 80% higher, at least about 100% higher, at least about 150% higher, or at least about 200% higher, than that of corresponding wild-type transgenic plants. In one aspect, such transgenic plants are characterized by a specific increase in the abundance of the free amino acids Arg, Pro, Lys, Val, Leu, or Trp.

In one aspect, such transgenic plants are further characterized by having a cyanide content in their root or tuber storage cells (measured after about seven months of growth) which is at least about 20% lower, at least about 40% lower, at least about 50% lower, at least about 60% lower, at least about 70% lower, at least about 80% lower, at least about 90% lower, or at least 95% lower than that of wild-type transgenic plants. In one aspect, such transgenic plants are characterized by a reduction in cyanide content of about 60 to 75% compared to that of wild-type plants. In one aspect, such transgenic plants are characterized by a cyanide content in their roots and tuber storage organs of approximately 20 to 50 μg/g fresh weight.

In one aspect, such transgenic plants are further characterized by having a cyanogenic glycoside content in their root or tuber storage cells (measured after about seven months of growth) which is at least about 20% lower, at least about 40% lower, at least about 50% lower, at least about 60% lower, at least about 70% lower, at least about 80% lower, at least about 90% lower, or at least 95% lower than that of wild-type transgenic plants. In one aspect, such transgenic plants are characterized by a reduction in cyanogenic glycoside content of about 50 to 75% compared to that of wild-type plants.

In one aspect such transgenic plants are further characterized by having an acetone cyanohydrin content in their root or tuber storage cells (measured after about seven months of growth) which is at least about 20% lower, at least about 40% lower, at least about 50% lower, at least about 60% lower, at least about 70% lower, at least about 80% lower, at least about 90% lower, or at least 95% lower than that of wild-type transgenic plants. In one aspect, such transgenic plants are characterized by a reduction in acetone cyanohydrin content of about 80 to 90% compared to that of wild-type plants. In one aspect, such transgenic plants are characterized by an acetone cyanohydrin content in their roots and tuber storage organs of approximately 0.5 to 2.0 μmoles/g fresh weight.

In one aspect, such transgenic plants are further characterized by having a linamarin content in their root or tuber storage cells (measured after about seven months of growth) which is at least about 20% lower, at least about 40% lower, at least about 50% lower, at least about 60% lower, at least about 70% lower, at least about 80% lower, at least about 90% lower, or at least 95% lower than that of wild-type transgenic plants. In one aspect, such transgenic plants are characterized by a reduction in linamarin content of about 50 to 75% compared to that of wild-type plants. In one aspect, such transgenic plants are characterized by a linamarin content in their roots and tuber storage organs of approximately 1.0 to 3.0 μmoles/g dry weight.

In any of these transgenic plant characteristics, it will be understood that the plants will be grown using standard growth conditions as disclosed in the Examples, and compared to the equivalent wild-type cultivar.

Because the expression vectors, promoters, and HNL genes of the present invention can function in a wide variety of plants, including monocots and dicots, a transgenic plant can be any type of plant which contains a promoter which is preferentially active in the root or tuber storage cells of the plant, and which can express the HNL in a chimeric gene containing the promoter.

Techniques for transforming a wide variety of plant species are well known and described in the technical and scientific literature. See, for example, Weising et al, (1988) Ann. Rev. Genet., 22:421-477. As described herein, a root-specific promoter is operably linked to the desired heterologous DNA sequence encoding a HNL in a suitable vector. The vector comprising a root-specific promoter fused to heterologous nucleic acid sequence encoding a HNL will typically contain a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorsulfuron or Basta. Such selective marker genes are useful in protocols for the production of transgenic plants.

DNA constructs containing a root-specific promoter linked to heterologous DNA encoding HNL can be introduced into the genome of the desired plant host by a variety of conventional techniques. For example, the DNA construct may be introduced directly into the DNA of the plant cell using techniques such as electroporation and microinjection of plant cell protoplasts. Alternatively, the DNA constructs can be introduced directly to plant tissue using biolistic methods, such as DNA micro-particle bombardment. In addition, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al., (1984) EMBO J., 3:2717-2722. Electroporation techniques are described in Fromm et al., (1985) Proc. Natl. Acad. Sci. USA, 82:5824. Biolistic transformation techniques are described in Klein et al., (1987) Nature 327:70-7. The full disclosures of all references cited are incorporated herein by reference.

A variation involves high velocity biolistic penetration by small particles with the nucleic acid either within the matrix of small beads or particles, or on the surface (Klein et al., (1987) Nature, 327:70-73,). Although typically only a single introduction of a new nucleic acid segment is required, this method particularly provides for multiple introductions.

Agrobacterium tumefaciens-meditated transformation techniques are well described in the scientific literature. See, for example Horsch et al., (1984) Science, 233:496-498, and Fraley et al., (1983) Proc. Natl. Acad. Sci. USA, 90:4803. See the Examples herein for a demonstration of the transformation of plant cells with a vector comprising a root-specific promoter driving the expression of HNL by Agrobacterium tumefaciens.

More specifically, a plant cell, an explant, a meristem, or a seed is infected with Agrobacterium tumefaciens transformed with the segment. Under appropriate conditions known in the art, the transformed plant cells are grown to form shoots and roots, and develop further into plants. The nucleic acid segments can be introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al., (1984) Science, 233:496-498; Fraley et al, (1983) Proc. Nat'l. Acad. Sci. U.S.A., 80:4803.

Ti plasmids contain two regions essential for the production of transformed cells. One of these, named transfer DNA (T DNA), induces tumor formation. The other, termed virulence region, is essential for the introduction of the T DNA into plants. The transfer DNA region, which transfers to the plant genome, can be increased in size by the insertion of the foreign nucleic acid sequence without its transferring ability being affected. By removing the tumor-causing genes so that they no longer interfere, the modified Ti plasmid can then be used as a vector for the transfer of the gene constructs of the invention into an appropriate plant cell, such being a “disabled Ti vector”.

All plant cells that can be transformed by Agrobacterium and whole plants regenerated from the transformed cells can also be transformed according to the invention so as to produce transformed whole plants that contain the transferred foreign nucleic acid sequence. There are various ways to transform plant cells with Agrobacterium, including: (1) co-cultivation of Agrobacterium with cultured isolated protoplasts, (2) co-cultivation of cells or tissues with Agrobacterium, or (3) transformation of seeds, apices, or meristems with Agrobacterium.

Method (1) requires an established culture system that allows culturing protoplasts and plant regeneration from cultured protoplasts. Method (2) requires (a) that the plant cells or tissues can be transformed by Agrobacterium and (b) that the transformed cells or tissues can be induced to regenerate into whole plants. Method (3) requires micropropagation.

In the binary system, to have infection, two plasmids are needed: a T-DNA containing plasmid and a vir plasmid. Any one of a number of T-DNA-containing plasmids can be used, the only requirement being that one be able to select independently for each of the two plasmids. After transformation of the plant cell or plant, those plant cells or plants transformed by the Ti plasmid so that the desired DNA segment is integrated can be selected by an appropriate phenotypic marker. These phenotypic markers include, but are not limited to, antibiotic resistance, herbicide resistance, or visual observation. Other phenotypic markers are known in the art and may be used in this invention.

The present invention embraces use of the claimed root-specific HNL expression constructs in transformation of any plant, including both dicots and monocots. Transformation of dicots is described in references above. Transformation of monocots is known using various techniques, including electroporation (e.g., Shimamoto et al., (1992) Nature, 338:274-276; ballistics (e.g., European Patent Application 270,356); and Agrobacterium (e.g., Bytebier et al., (1987) Proc. Nat'l Acad. Sci. USA, 84:5345-5349).

Transformed plant cells derived by any of the above transformation techniques can be cultured to regenerate a whole plant possessing the desired transformed phenotype. Such regeneration techniques rely on manipulation of certain phytohormones in a tissue culture growth medium typically relying on a biocide and/or herbicide marker that has been introduced together with the nucleotide sequences. Plant regeneration from cultured protoplasts is described in Evans et al., Handbook of Plant Cell Culture, pp. 124-176, MacMillan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally by Klee et al., Ann. Rev. Plant Phys., 38:467-486, 1987. Additional methods for producing a transgenic plant useful in the present invention are described in U.S. Pat. Nos. 5,188,642; 5,202,422; 5,463,175; and 5,639,947. Methods useful for cassava are disclosed in US. Pat. Nos. U.S. Pat. No. 7,072,836; U.S. Pat. No. 6,982,327 and U.S. Pat. No. 6,551,827, the disclosures of which are hereby incorporated by reference.

One of skill will recognize that, after a root-specific HNL expression cassette is stably incorporated in transgenic plants and confirmed to be operable, it can be introduced into other plants by sexual crossing. Any of a number of standard breeding techniques can be used, depending upon the species to be crossed.

The methods, compositions, and expression vectors of the present invention have use in a broad range of types of plants, including the creation of transgenic plant species belonging to the Fabaceae, Rosaceae, Gramineae, Euphorbiaceae, Olacsceae and Linaceae families. In one aspect, such transgenic plants are formed from wild-type plants that produce cyanogenic glycosides, and include for example, the following species: Cassava (Manihot esculenta), Sorghum (Sorghum vulgare), Flax (Linum usitatissimum), Lima beans (Phaseolus lunatus), Giant taro (Alocasia macrorrhizos), Bamboo (Bambusa arundinacea), Apple (Malus spp.), Peach (Prunus persica), Nectarine (Prunus persica var nucipersica), Cherry (Prunus spp.), Bitter almond (Prunus dulcis), raspberry, and crabapple.

### VI. Products & Uses

Another embodiment of the present invention includes processed foods, and starches and flours, made from cassava plants, obtained from the methods and transgenic plants of the present invention. In one aspect, such starches and flours are characterized by a higher protein content and reduced cyanogen content compared to starches and flours from the wild-type cassava. Accordingly, in one aspect, such flours and starches have a protein content at least about 20% higher, or about 30% higher, or about 40% higher, or about 50% higher, or about 100% higher, or about 200% higher, or more preferably at least about 300% higher, than flours and starches extracted from the wild-type cassava under similar conditions.

In another aspect, such flours and starches additionally have a cyanogen content that is at least about 20% lower, or about 30% lower, or about 40% lower, or about 50% lower, or about 60% lower, or about 70% lower, or about 80% lower, or more preferably at least about 90% lower, than flours and starches extracted from the wild-type cassava grown under similar conditions.

Cassava is a tuberous root of the Spurge family, Euphorbiaceae. As a fresh tuber, it is boiled in salted water and consumed directly or after further frying or baking. It is used in soups, stews, and the like, or it is mashed to a thick paste and fried. A variety of dried, pulverized products are known, including: a mash which is fermented, then dried to form a coarse, crunchy meal; the fibers are separated from the starch, which is dried and powdered. The cassava starch, also called cassava flour, is similar in properties to cornstarch. It has quite high expansion capabilities when mixed with water and gelatinized, and is therefore used as a thickener, an agent to increase the rise of many products, and an agent to improve consistency and homogeneity. There are many references to cassava starch or tapioca starch in the literature, and some references to cassava flour called tapioca flour. By their interchanging uses it is apparent that such uses generally refer to the starch product and not to the flour.

Prior to the instantly claimed invention, at least four flours of cassava were known. The two most common cassava flours are formed from cassava starch extraction processes: the starch and the extracted fiber mat. The third flour is a composite flour, i.e., a mixture of cassava flour and a high protein flour. The fourth flour is a whole flour of cassava.

Cassava starch, also called cassava flour, tapioca starch, and tapioca flour, is an extract of starch from cassava pulp that is dried and pulverized to a flour. Most literature references to cassava or tapioca flour are references to cassava starch. Cassava starch has been used as a substitute for up to 30% of the wheat flour content in wheat-based bread-type products, but it is not possible to substitute cassava starch for more than 30% of wheat in wheat-based baking products.

Cassava meal is a highly fibrous (often fermented) meal prepared from the dried pulp fiber by-product of cassava starch production. The particles of the meal are about ½-1 mm in diameter. Cassava meal is mixed with water and fried to produce a product called cassava bread. The bread is very hard and about ¼ inch thick. It exhibits no risen structure and is simply a hard mat of fibers. Other uses of the meal include mixing the meal with meats and gravies, preparation of a gruel, and sprinkling the meal over food.

Composite flours of cassava are combinations of cassava starch and high protein flours, such as peanut, soy, or wheat. Non-grain breads have been made from cassava composite flours. About a 30:70 ratio of high-protein flour to cassava starch is required, and chemical modifiers, fat, and sometimes malt are essential to successful preparation of the baked product.

Until the present invention, it was thought that the protein content of cassava flour and starch were too low to produce baked products of risen structure. Consistent with this hypothesis, it has not been possible to use cassava flour alone to produce non-wheat products of risen structure, and the risen structure-type products have only been possible from composite flours when chemical modifiers and fat are also used.

Other than the above-mentioned uses of cassava flour as an ingredient in baked goods, there have been very few attempts to develop food products from cassava flour. Pasta products have been prepared from composite flours containing cassava flour. Cassava starch is commonly used as a minor ingredient in ice cream.

In preparing the flour or starch, the cassava tubers are subjected to any preprocessing steps of washing, scrubbing, culling, rinsing, and the like, peeled by any techniques of the art; peeling while clean (not recycled) water is passing over the tubers is preferred, although cassava may also be processed unpeeled, rinsing (rinsing in distilled water is preferred, although may be omitted), comminuting, slicing, chopping, or any other techniques desired (although not necessary), preferably shredding; dehydrating the material by air drying (at any appropriate temperature), freeze drying, vacuum drying, or any other techniques or combination of techniques of the art, preferably air drying, and comminuting by such techniques as to produce a flour with a moisture content of less than about 5%.

In comminuting steps, the desired particle size is achieved while retaining most or all of the plant fiber and other non-farinaceous substance of the tuber. In one aspect, the flour product contains at least 50% of the plant fiber and other non-farinaceous substance of the tuber (which is defined to include woody ends, inner portions of the peel, and other woody portions that are not incorporated into cassava flour). In another aspect, the flour product contains at least 75% of the plant fiber and other non-farinaceous substance of the tuber. In another aspect, the flour product contains at least 90% of the plant fiber and other non-farinaceous substance of the tuber.

In one processing method (see PCT publication WO 2005/121183), the cassava roots are cut into parts, grated, and then formed into a slurry with water. The addition of water while the tubers are being grated optimizes the formation of a slurry during the grating process and aids in dissolving the undesired components (among which are minerals, proteins, and cyanides) to a larger extent than simply washing them away. The removal of undesired components from the slurry so as to obtain a product mass is achieved by filtering the slurry, with the product mass as the residue and the water containing the components that are undesired for the flour product as the filtrate.

Dry raw cassava may be processed to flour material. Thus, in one flour embodiment, dried peeled or unpeeled tubers are preferably peeled, and comminuted to a moderately fine to fine powder.

In another, embodiment, a cooked flour may be produced in the method above with the added step of heating by any means available to the art in processes prior to, during, or after, and in any combination with the processes listed above.

### EXAMPLES

### Methods

Plasmid Construction and Cassava Transformation

Plasmid pBI121, carrying cassava Hydroxynitrile Lyase (HNL) cDNA (White et al., (1998) Plant Physiol. 116: 1219-1225) cloned between the tuber specific patatin promoter and nos terminator, was PCR amplified by adding KpnI and PstI sites to the primers (HNL-F: 5′ GAGACTGCAGTTGTAGTTAATGCGTATTAGTTTTAGC 3′ (SEQ ID NO:1) and HNL-R: 5′ TCTCGGTACCGATCTAGTAACATAGATGACACCGCG 3′) (SEQ ID NO:2)), and the PCR amplified product was cloned at KpnI and PstI sites in pCambia2300. The arrangement of genes within the T-DNA region of pCambia 2300 vector from left border to right border is 2×CaMV35S: nptII: tNOS: patatin: HNL: mos. The modified binary vector was mobilized into Agrobacterium tumefaciens strain LBA4404 (Life Technology, Grand Island, N.Y., USA) by electroporation and used to transform cassava cultivar TMS60444 through a Friable Embryogenic Callus (FEC) system (Nigel Taylor, Personal Communication; Gonzalez et al. (1998) Plant Cell Reports 17:827-831).

PCR Analysis

To identify the presence of the transgene, PCR was carried out using the genomic DNA isolated from 45 d old in vitro plants. Total genomic DNA from leaf tissues was isolated using the DNeasy Plant Mini Kit from Qiagen (Qiagen Inc., Valencia, Calif., USA) according to the manufacturer's instructions. DNA from the untransformed plants was used as a negative control, and plasmid DNA of pCambia 2300 carrying Patatin-HNL-NOS were used as positive control. PCR analysis was performed employing gene-specific HNL primers (HNL F: 5′ AAGCTCAAACCAGCCCTTG 3′ (SEQ ID NO: 7) and HNL R: 5′ AATTTGCCAGCGTTGAAAGT 3′ (SEQ ID NO: 8)) and patatin primers (Pat-F: 5′ CGTCTCACAAAATTTTTAGTGACG 3′ (SEQ ID NO: 9) and Pat-R: 5′ TGATGTTTATTATCTCACTCACTTTGC 3′ (SEQ ID NO: 10)). The reaction mixture containing template, primers, buffer, dNTPs and Taq DNA polymerase was subjected to initial denaturation (94° C.) for 4 min, followed by repeated denaturation (94° C.) for 30 s, annealing (53° C.) for 30 s, and elongation (72° C.) for 1 min, for a total of 35 cycles. The final elongation step was carried out at (72° C.) for 10 min Amplified PCR products were analyzed by gel electrophoresis on 1.0% agarose gel.

Dot Blot Analysis

To identify the copy number of the transgenic plants, genomic DNA (100 ng) was used as template for dot blot. DNA was denatured with equal volume of 0.4 M NaOH by boiling for 5-10 min. and tubes were immediately placed onto ice for 5 min. 100 μl of 2×SSC were added to each DNA sample, and blotted to nylon membrane (Hybond N+) by using a BIO-DOT Micro filtration apparatus (Bio-Rad, Hercules, Calif.) according to the manufacturer's instructions. The membranes were washed with 2×SSC (300 mM NaCl, 30 mM sodium citrate), and DNA was crosslinked to membranes using a UV Stratalinker (auto-crosslink setting), and stored at room temperature. Three replicates for each DNA sample were maintained. TMS 60444 lines carrying CaMV 35S with 0, 1, 2, and 3 copies (a gift from Mohammed Abhary, Donald Danforth Plant Science Center, St. Louis, Mo.) were used as controls. 2×355 probe was amplified using 35S specific primers (35S-F: 5′ CACATCAATCCACTTGCTTTGAAG 3′ (SEQ ID NO: 11) and 355-R: 5′ CATGGTGGAGCACGACACT 3′ (SEQ ID NO: 12). Probe synthesis, hybridization, and washing were done using the DIG High prime DNA labeling and Detection Starter Kit II (Roche Applied Science, Indianapolis, Ind., USA) according to the manufacturer's instructions. The final detection was done by chemiluminescence (1:150) dilution of CDP-Star Reagent (Roche Diagnostics), followed by exposure to X-ray films. The films were scanned using Epscon Scanner and Spot finding; quantification and background subtraction were done using Image J (Image processing and analysis in Java). A standard curve equation was obtained between the copy numbers and spot intensities using the standard control plants carrying 0, 1, 2, and 3 copies of CaMV 35S. This equation was used to calculate the copy numbers in the transgenic lines.

Real Time-PCR Analysis

Total RNA from leaves and roots of 12 Patatin:HNL transgenic lines, WT-60444, and a CaMV 355-HNL transgenic line (Siritunga and Sayre., (2004) Plant Mol. Biol. 56: 661-669) was isolated using the RNA-easy kit from Qiagen (Qiagen Inc., Valencia, Calif., USA) according to the manufacturer's instructions. To remove contaminating genomic DNA, RNAs were treated with DNAase I (Promega, Madison, Wis., USA) according to the manufacturer's instructions. The concentrations of RNAs were assessed using a Nanodrop-2000C (Thermo-scientific, Wilmington, Del., USA) according to the manufacturer's instructions. The structural integrity of the RNAs was checked with non-denaturing agarose gel and ethidium bromide staining. DNase-treated RNA samples (0.5 μg) were reverse transcribed with an anchored oligo (dT) primer and 200 units superscript II reverse transcriptase (Invitrogen, Carlsbad, Calif., USA) in a volume of 20 μl according to the manufacturer's instructions. Real-time quantitative RT-PCR was carried out using an ABI-Step One Plus (Applied Biosystems, Foster City, Calif., USA) using PERFECTA™ SYBR® Green FASTMIX™ (ROX dye) (Quanta Biosciences, Gaithersburg, Md., USA) according to manufacturer's instructions. The cassava tubulin gene (Tub-F: 5′ GTGGAGGAACTGGTTCTGGA 3′ (SEQ ID NO: 3) and Tub-R: 5′ TGCACTCATCTGCATTCTCC 3′ (SEQ.ID. NO. 4) was used as reference gene/internal control and was amplified in parallel with the target HNL gene (HNL F: 5′ CAAACCAGCCCTTGAGAGAG 3′ (SEQ ID NO: 5) and HNL-R: 5′ TTCCCCTTGAGGGAGTTTCT 3′ (SEQ ID NO: 6), allowing gene expression normalization and providing quantification. Reactions were carried out with 50-100 ng/μl RNA in a final volume of 20 μl. All primers were designed using Primer Express software following the manufacturer's guidelines. For each sample, reactions were set up in quadruplicates, and two biological experiments were done to ensure the reproducibility of the results. The quantification of the relative transcript levels was performed using the comparative C_{T} (threshold cycle) method (Livak et al., (2001) Methods 25: 402-408).

Measurement of HNL Enzymatic Activity

Transformed and non-transformed cassava tissues (roots and leaves) from 5-month old plants (100 mg) were frozen in liquid nitrogen and ground in 0.5 ml of 0.05M sodium phosphate buffer, pH 5.0, 3 mM DTT, and 1% (w/v) polyvinyl pyrrolidine at 4° C. The cell wall material was pelleted by centrifugation at 13000 g for 15 min at 4° C. The supernatant was collected and centrifuged again to remove the cell debris. Supernatant protein concentrations were determined by CB-X™ Protein Assay (G-Biosciences, Maryland Heights, Mo., USA) according to manufacturer's instructions. Hydroxynitrile lyase assays were performed in a final volume of 1 mL containing 50 mM sodium phosphate buffer pH 5.0, 20 μg total leaf protein, and 28 mM acetone cyanohydrin (Sigma, St. Louis, Mo., USA), After 30 min incubation at 28° C. in capped tubes, 10 μL of the reaction mixture was added to 100 μl glacial acetic acid. 400 uL of reagent A (50 mg of succinimide and 125 mg N-Chlorosuccinimide in 50 mL water) and 400 uL of reagent B (3 g barbituric acid and 15 mL pyridine in 35 mL water) were added to the reaction mixture and incubated for 5 minutes. Enzyme activity was measured colorimetrically by measuring the absorbance at 585 nm. 10 μg/mL to 0.1 μg/mL of KCN was used as a standard to obtain a linear curve, and this equation was used to measure the amount of HL activity in transgenic lines.

Quantification of Protein Content

Cassava roots and leaves (10-15 mg) of 3, 5, and 7 month old transformed and non-transformed plants were homogenized in a mortar and pestle with protein extraction buffer (200 mM NaCl, 1 mM EDTA, 0.2% Triton-X, 100 mM Tris-HCl (pH 7.8), 4% 2-mercaptoethanol, supplemented with complete protease inhibitor cocktail (Roche, Basel, Switzerland). Tissues were extracted with 1 ml of extraction buffer with ceramic beads using a Fast Prep®-24 tissue and cell homogenizer (MP Biomedicals, Solon, Ohio, USA) at 5 m/s for 40 seconds. Samples were vortexed at 4° C. for 10 min a centrifuged at 9000 rpm for 10 min at 4° C., and supernatant was collected into a new tube. Extraction was repeated with another 1 ml extraction buffer. Supernatant protein concentrations were determined by CB-X™ Protein Assay (G-Biosciences, Maryland Heights, Mo., USA) according to manufacturer's instructions.

Quantification of Total Amino Acids and Free Amino Acids

Seven month old cassava transgenic and control lines (root tissues) were subjected to analysis for hydrolyzed and free amino acids. For hydrolyzed amino acids, samples were hydrolyzed for 24 h at 116° C. in 6N HCl containing 0.5% phenol. Samples were dried down and resuspended in 20 mM HCl, derivatized with the AccQ-tag reagent (Waters) and separated by ACQUITY UPLC® System (Waters, Milford, Mass., USA) according to manufacturer's instructions. Samples were prepared for free amino acid analysis according to Hacham et al. (2002) Plant Physiol. 128:454-462, and samples were subjected to ACQUITY UPLC® System (Waters, Milford, Mass., USA) according to manufacturer's instructions. Triplicates were maintained for each biological sample.

Western Blot

Five month old cassava transgenic and control lines were used for Western blot analyses. 5-20 μg of soluble protein were resuspended in 40 μl of sample buffer (0.06M Tris-HCl, pH 6.8, 10% (v/v) glycerol, 2% (w/v) SDS, 5% (v/v) 2-mercaptoethanol, 0.0025% (w/v) bromophenol blue) and heated at 95° C. for 5 minutes. Samples were centrifuged for 30 seconds at 15,000 g to remove debris, and samples were then separated by SDS-PAGE using 10% ready cast gels (Bio-Rad, Hercules, Calif., USA) at 20 mA for 3 hrs. Proteins were electrophoretically transferred onto a PVDF membrane using a semi-dry transfer apparatus at 1.9-2.5 mA/cm² of gel area for 60 minutes. The membrane was incubated for 1 hr in blocking solution (TBS: 20 mM Tris-HCl, pH 7.5, 500 mM NaCl, plus 0.5% BSA). Membranes were incubated for 24 hr at 4° C. with anti-HNL antibody diluted 1:1000 in TTBS containing 0.5% BSA (TBS containing 0.05% Tween-20). Membranes were washed three times with TTBS (15 min each), and secondary antibody (anti-rabbit IgG antibody (Sigma, St. Louis, Mo., USA) conjugated to horseradish peroxidase)) at 1:10000 dilution was added at room temperature for 2 h. Membranes were washed three times with TTBS (15 min each), and detection was performed by chemiluminescence using Luminal, Iodophenol, and Hydrogen Peroxide (Sigma, St. Louis, Mo., USA), followed by exposure to X-ray films (Yakunin et al., (1998) Analytical Biochemistry 258: 146-149).

Measurement of Hydrogen Cyanide Using Cyanide Sensing Electrode

Seven-month old transformed and control cassava lines were analyzed for hydrogen cyanide using a Cyanide Ion Selective Electrode (Thermo-Scientific, USA). Fresh or frozen root tissues (0.1 g) were extracted with 1 ml of 1×TBS buffer by adding ceramic beads using Fast Prep®-24 tissue and cell homogenizer (MP Biomedicals, Solon, Ohio, USA) 5 m/s for 40 seconds. Samples were incubated at RT for 10 minutes. Homogenates were transferred to 4 ml of 1×TBS buffer and 5 ml of 10M NaOH (final concentration of 5M). Samples were shaken gently at room temperature for 15 minutes and centrifuged at low speed for 1 minute to remove the cell debris. Samples were measured using a cyanide-specific electrode. A standard linear curve was prepared from a serial dilution of KCN (10⁻¹ to 10⁻⁵M).

Measurement of Free Cyanide and Acetone Cyanohydrin in Roots Following Maceration

Seven month old transgenic and control plants were used for cyanide and acetone cyanohydrin assays. Root cortex tissues (1 gram) were extracted with 5 mL of 0.1 M sodium phosphate buffer, pH 5.0, for 30 s by adding ceramic beads using FAST PREP®-24 tissue and cell homogenizer (MP Biomedicals, Solon, Ohio, USA) 5 m/s for 40 seconds, and incubated at 30° C. for 0-90 min in capped tubes. Starch was pelleted and removed by centrifugation at 7500 g for 2 min. The supernatant was immediately subjected to two assays. Liberated cyanide, a measure of acetone cyanohydrin decomposition, was measured by adding 0.5 mL of supernatant to 3.5 mL of 0.1 M sodium phosphate, pH 5.0, followed by cyanide quantification using a colorimetric method, as follows. A sample of the reaction mixture (100-500 μl) was added to 100 μl glacial acetic acid, 400 uL of reagent A (50 mg of succinimide and 125 mg N-Chlorosuccinimide in 50 mL water) and 400 uL of reagent B (3 g barbituric acid and 15 mL pyridine in 35 mL water). The reaction mixture was incubated for 5 minutes, and free cyanide was measured colorimetrically by measuring the absorbance at 585 nm. Total acetone cyanohydrin plus cyanide was determined by adding 0.1 mL of supernatant to 0.6 mL of 0.2 M NaOH and 3.3 mL of 0.1 mM sodium phosphate buffer, pH 5.0, followed by cyanide quantification using the colorimetric method described as above. The addition of NaOH converts all the acetone cyanohydrin into free cyanide. The amount of acetone cyanohydrin present in the HNL transformants was calculated as the difference between the two assays {(acetone cyanohydrin+cyanide assay)−(cyanide assay)}.

Measurement of Linamarin Content

Seven month old transgenic and control cassava lines were used to measure linamarin content. Both leaves and root tissues (10-25 mg dry weight) were used for solvent extraction. Acetonitrile (250 μl) was added to the dry lyophilized powder and extracted twice for 30 min by shaking. Samples were centrifuged and supernatants were transferred to a new tube. Supernatants were dried using a CentriVap DNA Concentrator (Labconco, Kansas City, Kans., USA) and dissolved with water, chloroform, and 10 μl of phenyl β-glucopyranoside (PGP). Samples were mixed, then centrifuged, and the upper phase was again dried using a CentriVap DNA Concentrator (Labconco, Kansas City, Kans., USA). Samples were re-dissolved with 50 μl of acetonitrile, and derivatized with 50 μl of MSTFA+1% TMCS (Pierce, Rockford, Ill., USA) and 10 μl of pyridine at 65° C. for 30 min on a dry heating block. GC-MS analysis was performed using an Agilent 5975C Series instrument (Agilent Technologies, Santa Clara, Calif., USA). A 30 meter long, 0.25 micron film thickness ZB-5MSi Zebron® Guardian with integrated guard capillary gas chromatography column (#7HG-G018-11-GGA; Agilent Technologies, Santa Clara, Calif., USA) at an injection temperature of 250° C. was used for separation. The GC-MS was operated under a pressure control mode using pressures that gave flow rates near 1 mL/minute. The GC oven temperature program was: 50° C. for one minute after injection, ramp at 30° C./minute to 185° C., ramp at 6° C./minute to 230° C. (linamarin elution), ramp at 12° C./minute to 280° C. (internal standard elution), and 3 minutes at 290° C. to clean the column Standards were prepared with different concentrations of internal standard, phenyl β-glucopyranoside (PGP), and linamarin (Sigma). The peak areas of linamarin and PGP were plotted to obtain a linear curve and this equation was used to measure the amount of linamarin in transgenic and control lines.

### Example 1

### Molecular Characterization of Transgenic Plants Expressing HNL from the Patatin Promoter

Patatin has been widely used as a tissue-specific promoter, including class I-patatin promoter (CIPP) (Bevan et al., (1986) Nucl. Acids Res. 14: 4625-4637; Jefferson et al., (1990) Plant Mol. Biol. 14: 995-1006; Grierson et al., (1994) Plant J. 5: 815-826). This tuber-specific promoter has been used to control the expression of transgenes in several crops including potato and cassava (Zhu et al., (2008) Plant Cell Rep. 27: 47-55; Ihemere et al., (2006) Plant Biotechnol. J. 4: 453-465). To determine the feasibility of using this promoter to drive HNL expression in cassava, the endogenous cassava Hydroxynitrile Lyase (HNL) coding region was cloned between the patatin promoter and nos terminator, and introduced into cassava cultivar TMS60444 by Agrobacterium tumefaciens transformation, as described above.

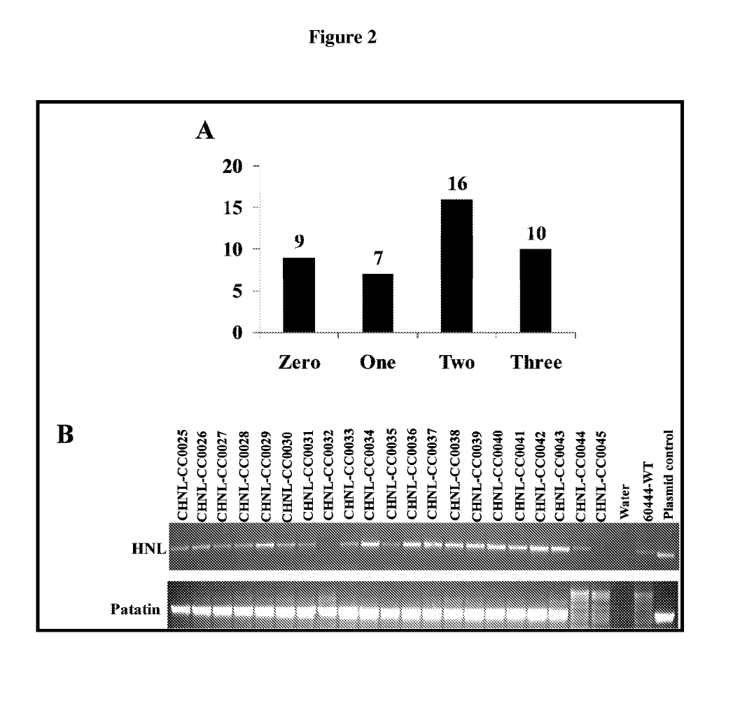

Using a high-throughput Friable Embryogenic Calli (FEC's) transformation system (Gonzalez et al. (1998) Plant Cell Reports 17:827-831) forty-two independent transgenic lines were obtained, and 33 lines showed the presence of the HNL gene by molecular analysis (FIG. 2). In order to determine the copy number of the HNL transgenic lines, dot blot analysis was conducted. Scanning and analysis of the blots using digital image analysis indicated that seven lines showed one copy, sixteen lines showed two copies, and ten lines showed three copies, of the gene (FIG. 2A). To verify the presence of the HNL gene and patatin promoter, PCR analyses of leaf tissues were performed. A sample of PCR results obtained with 21 transgenic lines and wild-type (60444) are shown in FIG. 2B. Since there is endogenous HNL expressed in leaves, the wild-type (60444) lines also show expression of the endogenous HNL (FIG. 2B). Therefore, to verify the presence and abundance of the gene, quantitative PCR was performed both in leaves and roots of transgenic HNL lines and wild-type plants.

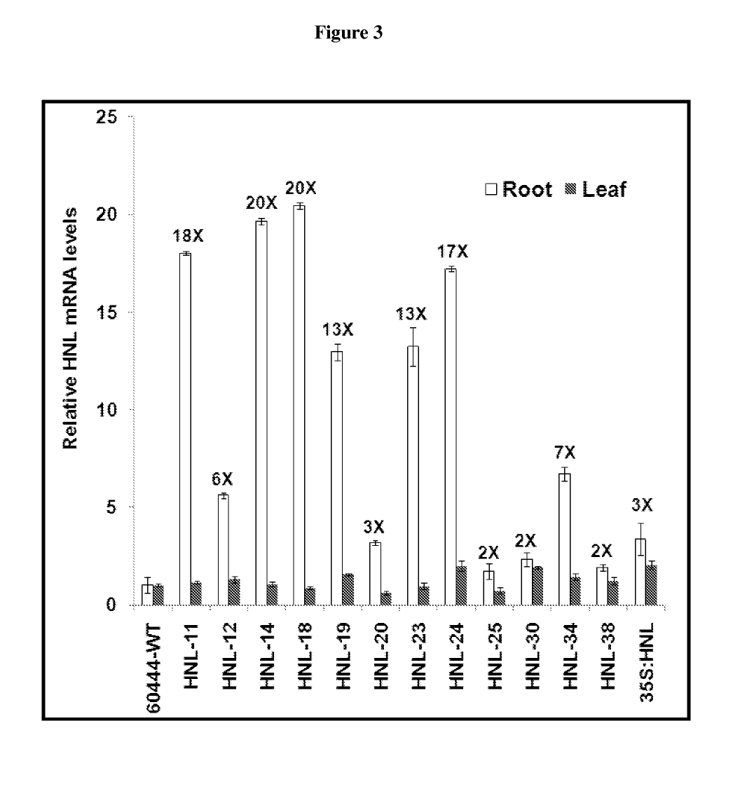

To calculate the relative expression of HNL in transgenic lines, all expression values were normalized relative to tubulin to adjust for differential loading, and to the expression of the endogenous HNL in WT-60444, which was taken as a calibrator and its expression value adjusted to one. Transgenic lines carrying HNL showed a varied range of increase (2-20 fold) of relative mRNA expression in roots when compared with wild-type, and a 5-6 fold increase when compared with a CaMV 35S HNL transgenic line (FIG. 3). Among the twelve independent transgenic lines analyzed, several lines, including HNL-11, HNL-14, HNL-18, and HNL-24, showed very high expression of HNL mRNA, while a few lines, including HNL-20, HNL-25, HNL-30, and HNL-38, showed only a 2-3 fold increase, when compared with the wild-type (FIG. 3). There was no significant difference in mRNA expression found in the leaves between the transgenics and the wild-type (FIG. 3). Interestingly, the CaMV 35S:HNL transgenic line showed a slight increase of HNL expression in the leaves when compared with the patatin: HNL transgenic lines (FIG. 3).

Conclusions:

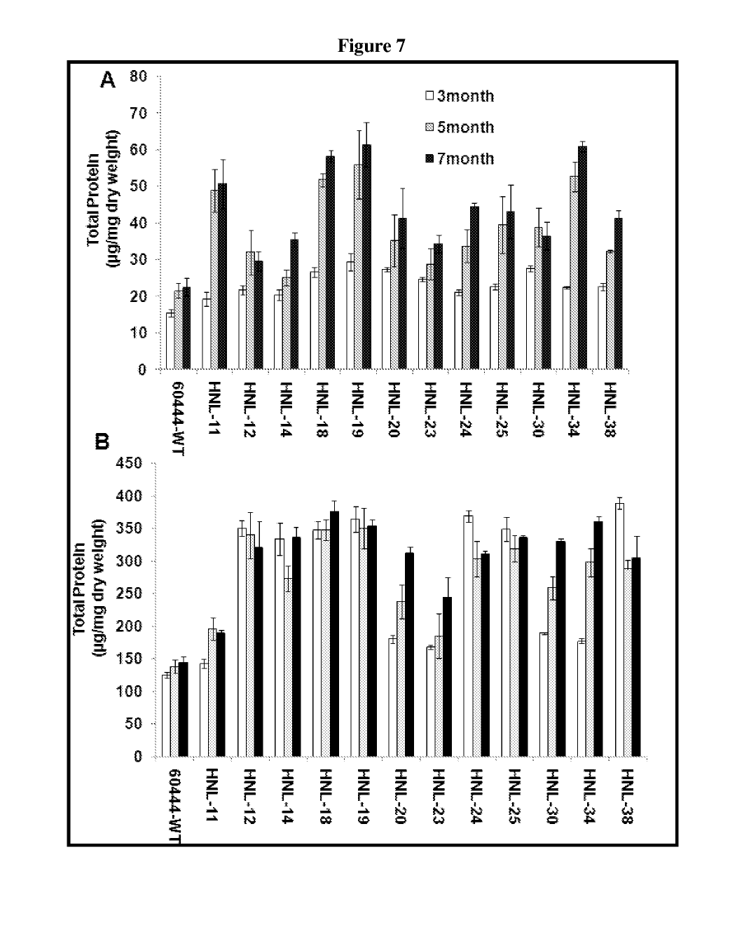

These results clearly demonstrate that overexpression of HNL using the patatin promoter results in a 5-6 fold increase in relative mRNA expression in roots when compared with 35S promoter transgenic lines (FIG. 3). Similar results were obtained when CrtB (genes encoding Phytoene Synthase) were overexpressed in potato using tuber-specific patatin promoters (Diretto et al. (2010) Plant Physiol. 154:899-912). Quantitative PCR analyses revealed low, but detectable, CrtB transcript levels in leaves of the transgenic lines, suggesting that patatin promoters allowed low levels of expression in leaves (Diretto et al. (2010), supra). The gradual increase of total protein concentrations in 3-7 month old plants is a clear indication of the tuber-specific patatin promoter and its effects (FIG. 7A).

### Example 2

### Measurement of the Specific Activity of Hydroxynitrile Lyase Increases in Transgenic Roots

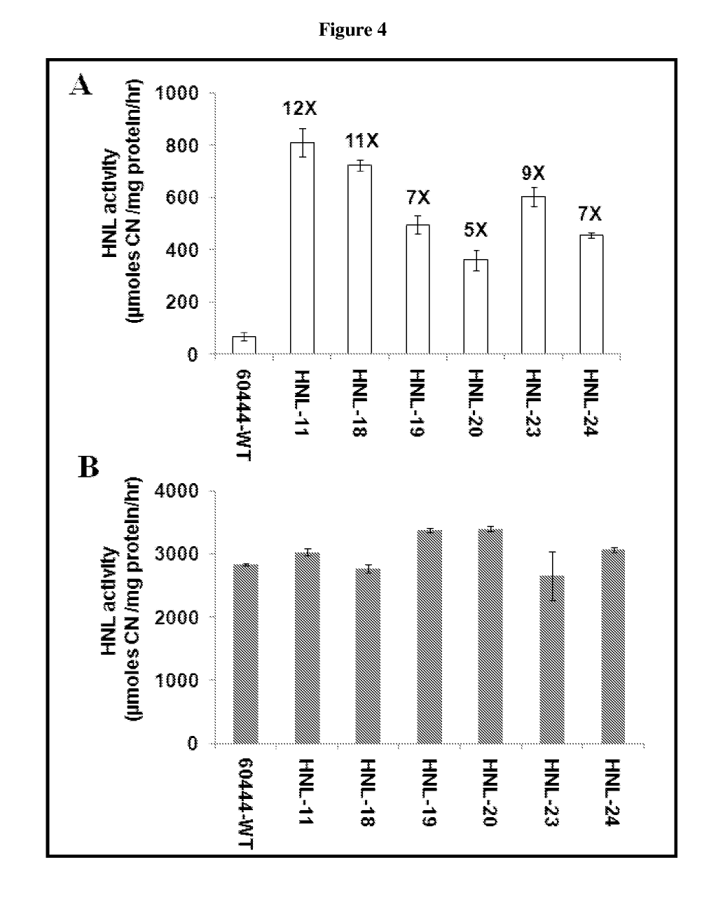

HNL enzyme activity was measured in both roots and leaves of transgenic and wild-type (60444) cassava lines. Six independent transgenic lines (HNL-11, HNL-18, HNL-19, HNL-20, HNL-23, and HNL-24) were selected for analysis. Analysis of the HNL activity of roots indicated that there was a large variation in increase in enzyme rates between the transgenic clones and the wild-type (FIG. 4A). In the transformed cassava lines (HNL-11, HNL-18, HNL-19, HNL-20, HNL-23, and HNL-24), HNL specific activity was 810.81, 732.91, 495.93, 360.98, 602.05, and 455.04 moles HCN/mg protein/h, respectively, compared with 68.59 μmoles HCN/mg protein/h in the wild-type. There was also a dramatic variation in increase from 5-fold (HNL-20) to 12-fold (HNL-11) in enzyme activity when compared with the wild-type. There were no significant differences observed in the enzyme rates in leaves between the transgenics and wild-type (FIG. 4B).

Conclusions:

In this study, there was a 5- to 12-fold increase of HNL enzyme activity in transgenic roots when compared with that in the wild-type (FIG. 4A). Previously, an 8- to 13-fold increase in root HNL activity in transformed plants (35S: HNL) relative to wild type was observed (Siritunga and Sayre, (2004) Plant Mol. Biol. 56: 661-669). These differences in root HNL activity between the two studies may be due to the sensitivity of the techniques used in these studies. It is important to note that there is no significant increase of HNL activity in transgenic leaves when compared with the wild-type, suggesting the effect of the tuber-specific promoter (FIG. 4A). Root HNL activities in transgenic plants are substantially less than those in wild-type leaves, suggesting the presence of high HNL in leaves when compared with the roots (White et al., (1998) Plant Physiol. 116: 1219-1225).

### Example 3

### Western Blot Analysis of Protein Expression Levels

To establish whether the observed increase in root HNL activity was correlated with the greater HNL protein abundance, protein blots were carried out for transgenic and control root and leaf tissues.

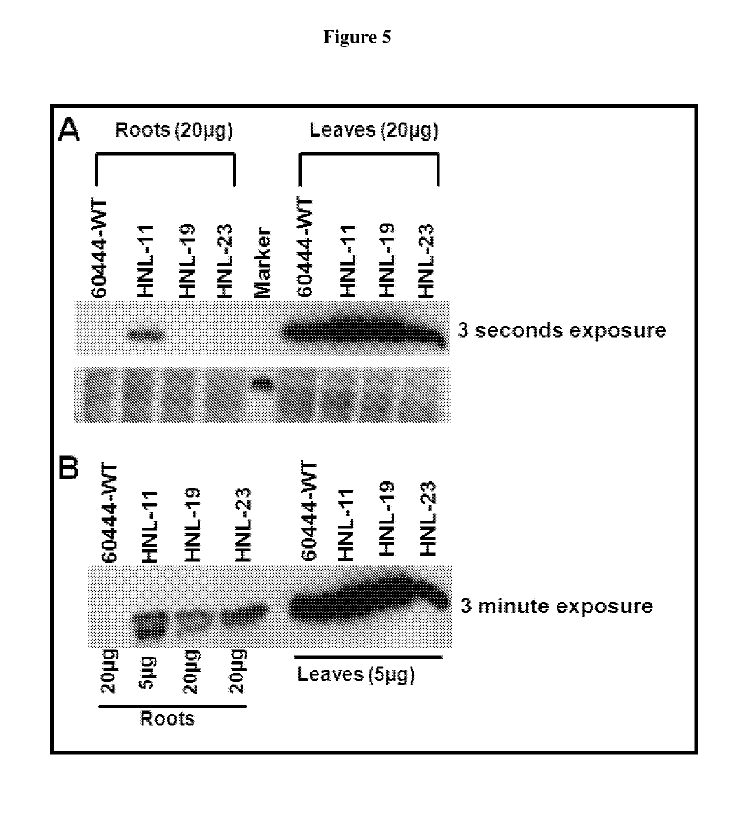

To determine the relative levels of HNL in roots using the patatin promoter, both transgenic and wild-type roots were subjected to Western blot analysis. Within 3 seconds of exposure, none of the root samples except HNL-11 showed an immunodetectable band. Interestingly, root samples of HNL-11 showed a clear band suggesting that it is a higher expresser (FIG. 5A).

In order to determine the relative expression levels of HNL in transgenic plants, immunoblots were loaded with different amounts of protein as indicated in FIG. 5B. Within 3 minutes of exposure with the detection system, both HNL-19 and HNL-23 showed the presence of HNL protein, while wild-type roots did not show the presence of specific HNL protein. Even with 5 μg of protein, root samples of HNL-11 and all the leaf samples studied showed a clear, distinct band, suggesting higher expression levels of HNL (FIG. 5B).

It is has been previously demonstrated that HNL was not detectable in wild-type cassava roots by Western blot analysis using polyclonal antibodies generated in mice (White et al., (1998) Plant Physiol. 116: 1219-1225). In this study, HNL-11, which is considered to be an overexpresser, showed a 29 kDa HNL protein band within 3 seconds of exposure to the detection system (FIG. 5A). Based on the fact that there is a linear correlation between the amount of HNL protein detected and the antibody titer used for protein blots (White et al., (1998) Plant Physiol. 116: 1219-1225), the data suggest that there is a strong correlation between HNL enzyme activity and protein abundance in transgenic roots.

### Example 4

### Measurement of Cyanide Levels in Transgenic Roots

Strategies for reducing cyanogen toxicity in cassava have been carried out either by blocking the synthesis of linamarin (Siritunga and Sayre, (2003) Planta, 217: 367-373; Jorgensen et al., (2005) Plant Physiol. 139: 363-374), or by accelerating cyanogenesis and cyanide volatilization (Siritunga and Sayre, (2004) Plant Mol. Biol. 56: 661-669). It has been found that the most abundant cyanogen in poorly processed cassava is acetone cyanohydrin, which is the substrate for HNL (Tylleskar et al., (1992) Lancet. 339: 208-211). Since it was apparent that the lack of HNL in cassava roots could lead to the accumulation of acetone cyanohydrin, overexpression of HNL would be expected to accelerate cyanogenesis, increase CN volatilization, and therefore lead to an overall reduction in cyanide toxicity. To determine whether the overexpression of HNL in transgenic plants using the patatin promoter enhanced root cyanogenesis, we measured cyanide, acetone cyanohydrins, and linamarin levels in transgenic roots.

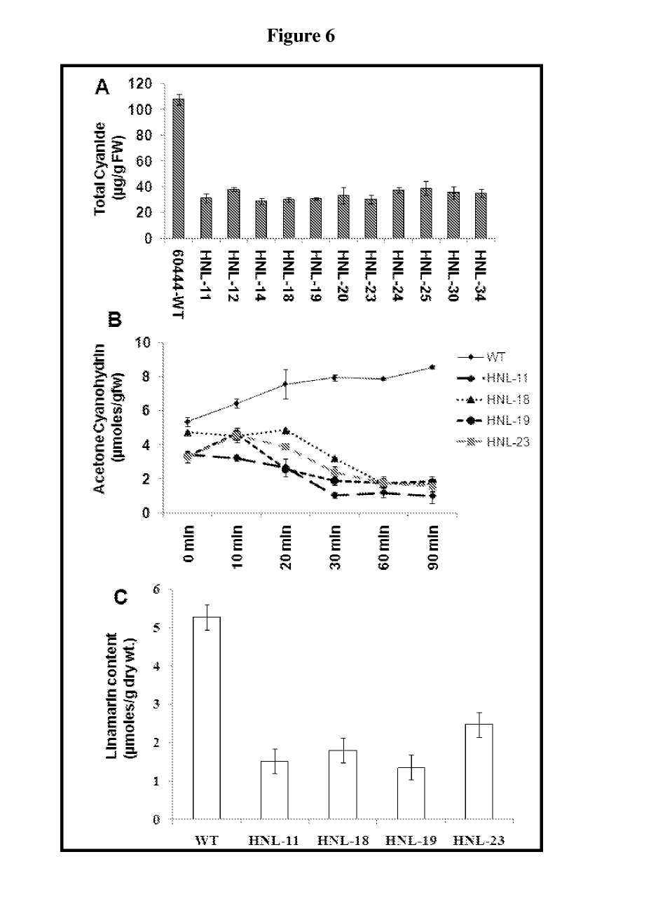

Seven month old root tissues of twelve independent transgenic lines and the wild-type cassava were analyzed to determine their cyanide content, as described above. Transgenic lines displayed a range of 60-75% reduction in cyanide when compared with the wild-type plants (FIG. 6A). All the transgenics showed dramatic reduction in cyanide concentrations (ranging from 29-40 μg/g fresh weight) when compared with the wild-type (107.75 μg/g fresh weight).

To determine whether the overexpression of HNL in transgenic plants enhanced root cyanogenesis, we measured residual acetone cyanohydrin and cyanide in homogenized roots as a function of incubation time post-homogenization. As shown in FIG. 6B, roots from transformed plants having elevated levels of HNL in roots had an 80-90% reduction in acetone cyanohydrin.

The transgenic line HNL-11 (highest expresser) showed very low levels of acetone cyanohydrins (0.99 μmoles/g fresh weight) compared to roots from control plants (8.56 μmoles/g fresh weight) following 90 min of incubation, post-homogenization (FIG. 6B). The other three lines studied (HNL-18, HNL-19, and HNL-23) also showed lower levels of acetone cyanohydrin (1.7, 1.83, and 1.57 μmoles/g fresh weight, respectively) as shown in FIG. 6B.

To determine the pool sizes of linamarin in roots and study the effect of over-expression of HNL, linamarin content in both leaves and roots was measured. Significantly, the average steady state linamarin content of leaves from control and transgenic lines studied was nearly identical (ranging from 25-30 μmoles/g dry weight; data not shown). Interestingly, the average steady state linamarin content of roots in transgenic lines showed 53-74% reduction when compared with that of the wild-type plants (FIG. 6C). HNL-19 showed very low levels (1.36 μmoles/g dry weight) compared to roots from control plants (5.26 μmoles/g dry weight) (FIG. 6B). The other three lines studied (HNL-11, HNL-18, and HNL-23) also showed lower levels of linamarin (1.52, 1.8, and 2.47 μmoles/g dry weight, respectively), as shown in FIG. 6C.

Conclusions:

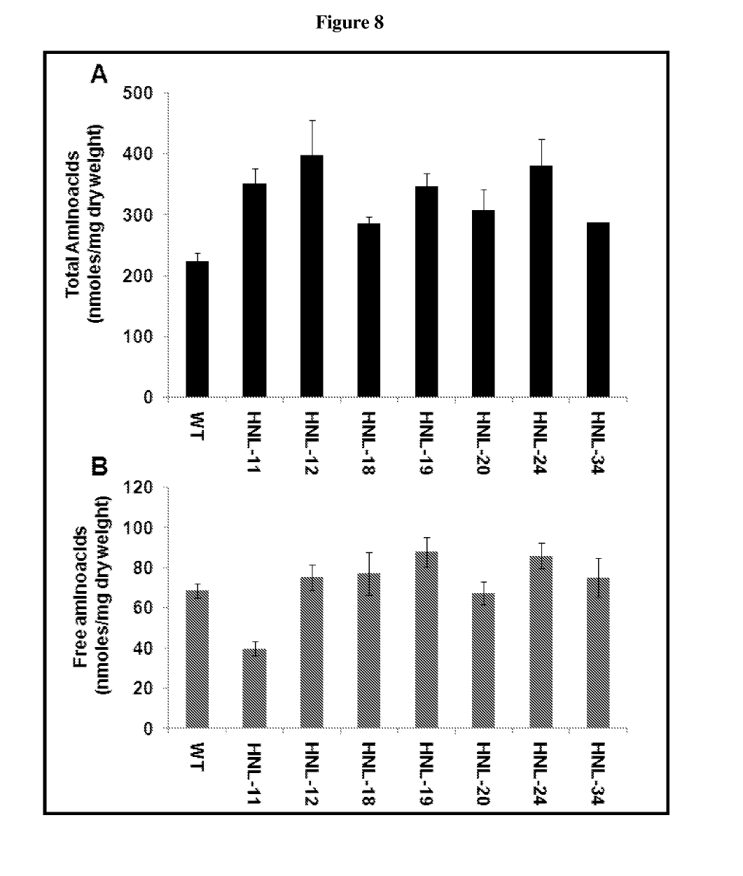

After processing and post-homogenization, transgenic lines displayed a range of 60-75% reduction in cyanide (FIG. 8A) and 80-90% reduction in acetone cyanohydrin (FIG. 8B) when compared with the wild-type plants. These results clearly suggest that transgenic lines overexpressing HNL enhanced root cyanogenesis, and show reduced cyanogen levels. It was observed that 80-90% of the linamarin was converted to acetone cyanohydrin within 90 min. of incubation, post-homogenization. HNL-11, expressing 12-fold higher HNL activity, had almost 90% reduction in acetone cyanohydrin when compared with the wild-type plants, suggesting that the presence of elevated amounts of HNL enzyme in the transgenic roots resulted in the accelerated turnover of acetone cyanohydrin compared with that in the wild-type plants (FIG. 8B).

### Example 5

### Measurement of Total Protein Concentrations

Twelve transformants (HNL-11, HNL-12, HNL-14, HNL-18, HNL-19, HNL-20, HNL-23, HNL-24, HNL-25, HNL-30, HNL-34, and HNL-38) and control line 60444 were subjected to protein analysis by using CB-X assay kit as explained in Methods. Both roots and leaf tissues of three, five, and seven month old cassava plants were used in this study.

HNL transgenics showed higher (2-3 fold) protein concentrations in roots compared with that in the wild-type plants (FIG. 7A). In 7 month old plants, HNL-19 showed the highest protein concentrations in roots (61.37 μg/mg dry weight) when compared with the wild type plants (22.38 μg/mg dry weight) (FIG. 7A). Furthermore, compared with control plants, all the transgenics showed enhanced increase in protein concentrations (ranging from 29-61 μg/mg dry weight). Transgenic lines also exhibited enhanced protein increase over time from 3-7 months when compared with that in the wild-type plants. It is interesting to note that transgenic lines such as HNL-11 (19.26 to 50.61 μg/mg dry weight), HNL-19 (29.25 to 61.37 μg/mg dry weight), and HNL-34 (22.38 to 60.86 μg/mg dry weight) showed almost 2-3 fold increase from 3-7 months, suggesting the effect of the tuber-specific patatin promoter (FIG. 6A). Similarly, transgenic lines also showed higher protein concentrations in leaves (2-3 fold) when compared with those in the wild-type plants (FIG. 7B). Most of the transgenic clones displayed higher increases in protein content when 3-7 months old. In 7 month old leaves, among the twelve lines studied, HNL-18 and HNL-19 showed the highest protein concentrations (375 and 353 μg/mg dry weight) when compared with those in the wild-type plants (144 μg/mg dry weight) (FIG. 7B).

### Example 6

### Measurement of Total Amino Acids and Free Amino Acids

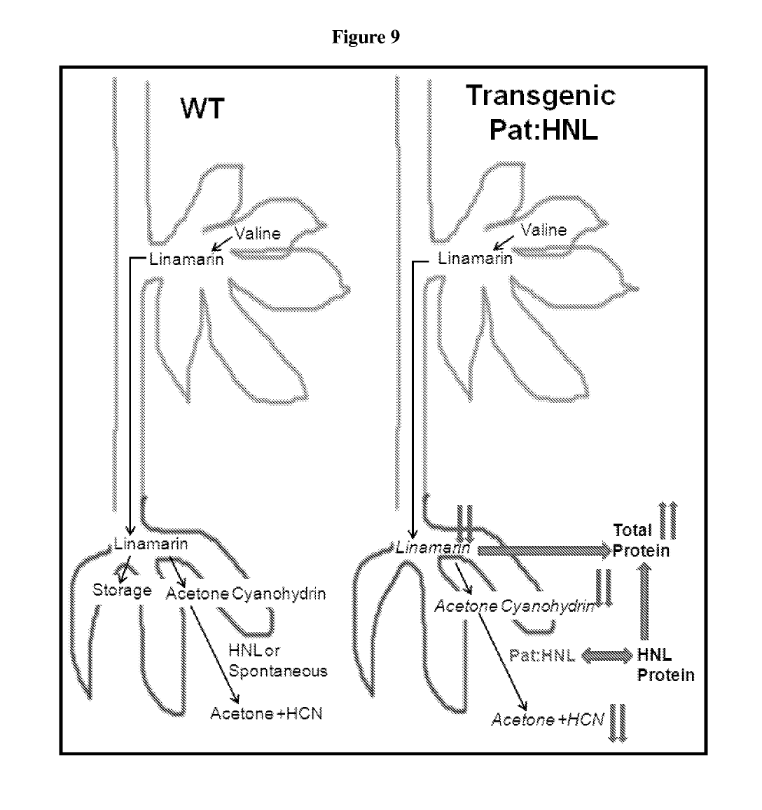

Seven month old root tissues of transgenic lines (HNL-11, HNL-12, HNL-18, HNL-19, HNL-20, HNL-24, and HNL-34) and wild-type were tested for total and free amino acid content. The results of this analysis with respect to the level of total amino acids in these different transgenic lines are shown in FIG. 8A, and individual amino acid composition is shown in FIG. 9. Transgenic lines exhibited about a 1.5-2 fold increase in total amino acids, correlating with the increase in protein concentrations.

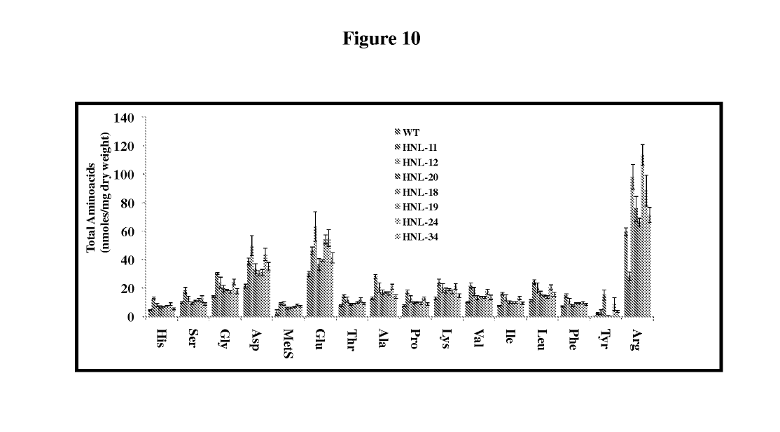

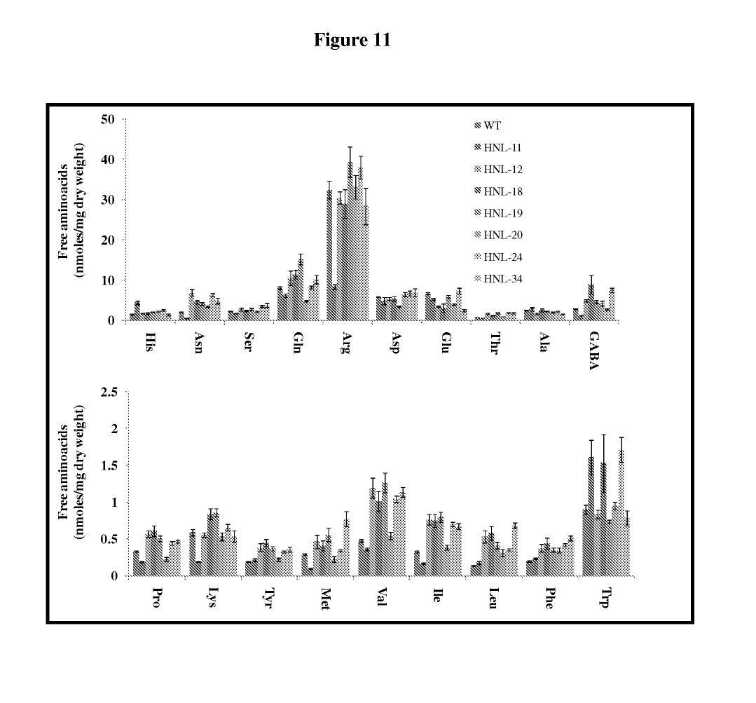

In most of the transgenic lines analyzed, the most abundant amino acids were GLY, ASP, GLU, and ARG (FIG. 9). Other amino acids, such as HIS, SER, THR, ALA, PRO, LYS, VAL, ILEU, and LEU also showed an increase in the transgenic lines compared with the wild-type (FIG. 9). There were no significant differences in the free amino acids between the transgenics and wild-type except for HNL-11 (FIGS. 8B and 10). Interestingly, in this transgenic line alone, a net decrease in the level of ARG was observed compared with that in the wild-type cassava. This reduction in total free amino acids in transgenic line HNL-11 is mainly due to slight decreases in certain amino acids such as ASN, SER, ARG, ASP, GLU, PRO, LYS, and ILEU (FIG. 10). In the other transgenic lines, the most abundant free amino acids were ASN, PRO, VAL, ILEU, LEU, and TRP; however, increases in these amino acids did not significantly increase the total free amino acid pool in the transgenic clones when compared with that in the wild-type plants (FIG. 11).

Cyanogenic glucosides have formerly been proposed to serve an important role as reservoirs of reduced nitrogen (Selmar et al., (1988) Plant Physiol. 86: 711-716). This hypothesis was initially proposed in the endosperm (85% of the seed dry matter) of rubber seeds, which contains more than 90% of linamarin. It was found that during germination and plantlet development, 85% of the cyanogenic potential of the entire seedling declines, with negligible amounts of gaseous HCN liberated during this process. Due to the fact that the detoxifying enzyme β-cyanoalanine synthase is present in high levels in young seedling tissues, it was proposed that linamarin is transported from the endosperm via the apoplast to the young growing tissues (Selmar et al., (1988) Plant Physiol. 86: 711-716), where it is assimilated into amino acids. There are also reports suggesting that the closely related derivative of linamarin, linustatin, also moves via the apoplast and vascular system to target tissues, where it is degraded to HCN (Poulton., (1990) Plant Physiol. 94: 401-405).

In cassava, it has also been proposed that linamarin may be used as a transportable source of reduced nitrogen for amino acid synthesis in roots (Siritunga and Sayre, (2007) JAOAC Int. 90: 1450-1455). The conversion of cyanide to asparagine has been well demonstrated using radiolabelled precursors in sorghum and cassava (Nartey, (1969) Physiologia plantarum. 22: 1085-1096). In this work, the average linamarin content of roots in transgenic lines showed 53-74% reduction when compared with that of the wild-type plants (FIG. 8C). It is also important to note that linamarin levels in leaves did not significantly change between the wild-type and the transgenic plants, suggesting that this could be a favorable trait for farmers due to their generalist herbivore deterrent qualities.

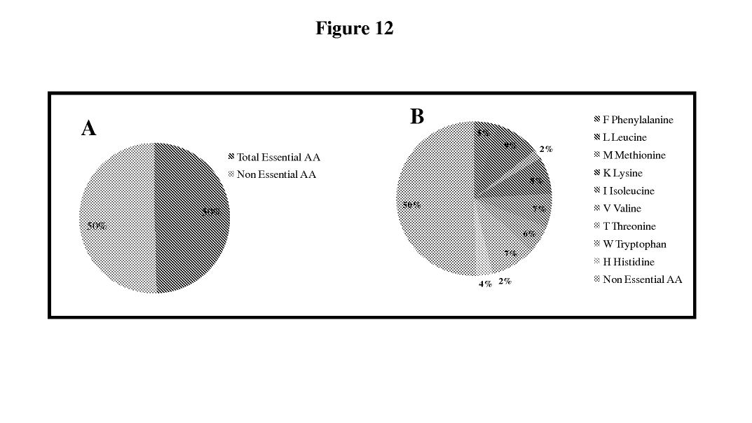

At the same time, we also observed that overexpression of HNL leads to a 2-3 fold increase in total protein concentrations in the transgenic roots and leaves when compared with the wild-type plants (FIG. 7), suggesting that there is a strong contribution of linamarin metabolism to root nitrogen balance. We also show that there is a 2-fold increase in root total amino acids in the transgenic plants, and no significant increase in free amino acids when compared with that in the wild-type, suggesting that the increased protein level is a reflection of the accumulation of HNL protein (FIG. 8), which ultimately correlates with increase in total protein in transgenic cassava roots (FIG. 12). Essential amino acids such as Lysine (Lys), leucine (Leu), methionine (Met), and valine (Val) are known to contribute substantially to the nutritional quality of any crop plants (Ufaz ad Galili, (2008) Plant Physiol. 147: 954-961).

Conclusions:

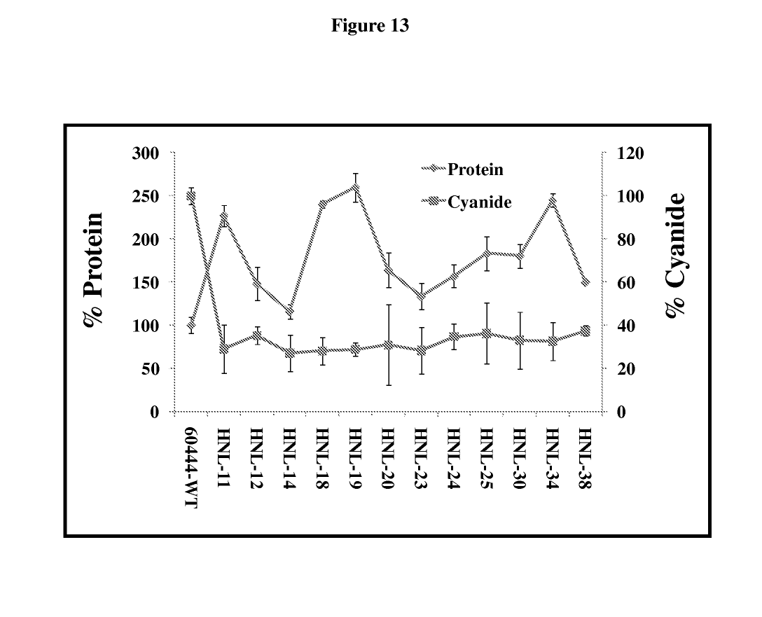

We have shown a dual role of cassava HNL and a direct correlation between decreasing cyanide, and increasing protein, concentrations in roots (FIG. 13).

Without being bound by any one particular theory of operation, it is speculated that there is a strong contribution of linamarin metabolism from the leaves to root protein synthesis (FIG. 12).

Using cassava as a model system, the effects of overexpressing HNL on amino acid metabolism was studied for the first time. Overall, these results demonstrate that the over-expression of HNL in cassava roots accelerates root cyanogenesis and increases the protein content of the roots. Moreover, these cassava roots will have reduced cyanogens and increased protein levels compared to those in wild-type plants, which will make cassava a nutritionally biofortified crop, as well as provide a safer food product.

SEQ ID Summary

| SEQ ID NO: | Sequence | Organism |
|---|---|---|
| SEQ.ID.NO. 1 | GAGACTGCAGTTGTAGTTAATGCGTATTAGTTTTAGC | Synthetic |
| SEQ ID NO: 2 | TCTCGGTACCGATCTAGTAACATAGATGACACCGCG | Synthetic |
| SEQ ID NO: 3 | GTGGAGGAACTGGTTCTGGA | Synthetic |
| SEQ.ID.NO. 4 | TGCACTCATCTGCATTCTCC | Synthetic |
| SEQ ID NO: 5 | CAAACCAGCCCTTGAGAGAG | Synthetic |
| SEQ ID NO: 6 | TTCCCCTTGAGGGAGTTTCT | Synthetic |
| SEQ ID NO: 7 | AAGCTCAAACCAGCCCTTG | Synthetic |
| SEQ ID NO: 8 | AATTTGCCAGCGTTGAAAGT | Synthetic |
| SEQ ID NO: 9 | CGTCTCACAAAATTTTTAGTGACG | Synthetic |
| SEQ ID NO: 10 | TGATGTTTATTATCTCACTCACTTTGC | Synthetic |
| SEQ ID NO: 11 | CACATCAATCCACTTGCTTTGAAG | Synthetic |
| SEQ ID NO: 12 | CATGGTGGAGCACGACACT | Synthetic |
| SEQ ID NO: 13 | TTGTAGTTAA TGCGTATTAG TTTTAGCGAC GAAGCACTAA | Potato |
| | ATCGTCTTTG TATACTTTGAGTGACACATG TTTAGTGACG | |
| | ACTGATTGAC GAAATTTTTT TCGTCTCACA AAATTTTTAG | |
| | TGACGAAACA TGATTTATAG ATGACGAAAT TATTTGTCCC | |
| | TCATAATCTA ATTTGTTGTAGTGATCATTA CTCCTTTGTT | |
| | TGTTTTATTT GTCATGTTAG TTCATTAAAA AAAAAATCTC | |
| | TCTTCTTATC AATCCTGACG TGTTTAATAT CATAAGATTA | |
| | AAAAATATTT TAATATATCTTTAATTTAAA CTCACAAAGT | |
| | TTAATTTTCT TCGTTAACTT AATTTGTCAA ATCAGGCTCA | |
| | AAGATCGTTT TTCATATCGG AATGAGGATT TTATTTATTC | |
| | TTTTAAAAAT AAAGAGGTGTTGAGCTAAAC AATTTCAAAT | |
| | CTCATCACAC ATATGGGGTC AGCCACAAAA ATAAAGAACG | |
| | GTTGGAACGG ATCTATTATA TAATACTAAT AAAGAATAGA | |
| | AAAAGGAAAG TGAGTGAGGTGCGAGGGAGA GAATCTGTTT | |
| | AATATCAGAG TCGATCATGT GTCAGTTTTA TCGATATGAC | |
| | TTTGACTTCA ACTGAGTTTA AGCAATTCTG ATAAGGCGAG | |
| | GAAAATCACA GTGCTGAATCTAGAAAAATC TTATACAATG | |
| | TGAGATAAAT CTCAACAAAA ACGTTGAGTC CATAGAGGGG | |
| | GTGTATGTGA CACCCCAACC TCAGCAAAAG AAAACCTCCC | |
| | CTCAAGAAGG ACATTTGCGGTGCTAAACAA TTTCAAGTCT | |
| | CATCACACAT ATATATTATA TAATACTAAT AAAGAATAGA | |
| | AAAAGGAAAG GTAAACATCA CTAATGACAG TTGCGGTGCA | |
| | AAGTGAGTGA GATAATAAACATCAGTAATA GACATCACTA | |
| | ACTTTTATTG GTTATGTCAA ACTCAAAATA AAATTTCTCA | |
| | ACTTGTTTAC GTGCCTATAT ATACCATGCT TGTTATATG | |
| SEQ ID NO: 14 | Gly-X-Ser/Cys-X-Gly/Ala-Gly/Ala | Synthetic |
| SEQ.ID.NO. 15 | MVTAHFVLIH TICHGAWIWH KLKPALERAG HKVTALDMAA | Manihot |
| | SGIDPRQIEQ INSFDEYSEP LLTFLEKLPQ GEKVIIVGES | esculenta |
| | CAGLNIAIAA DRYVDKIAAG VFHNSLLPDT VHSPSYTVEK | |
| | LLESLPDWRD TEYFTFTNIT GETITTMKLG FVLLRENLFT | |
| | KCTDGEYELA KMVMRKGSLF QNVLAQRPKF TEKGYGSIKK | |
| | VYIWTDQDKV FLPDFQRWQI ANYKPDKAYQ VQGGDHKLQL | |
| | TKTEEVAHIL QEVADAYA | |
| SEQ.ID.NO. 16 | MAFAHFVLIHT ICHGAWIWHK LKPLLEALGH KVTALDLAAS | Hevea |
| | GVDPRQIEEI GSFDEYSEPL LTFLEALPPG EKVILVGESC | Brasiliensis |
| | GGLNIAIAAD KYCEKIAAAV FHNSVLPDTE HCPSYVVDKL | |
| | MEVFPDWKDT TYFTYTKDGK EITGLKLGFT LLRENLYTLC | |
| | GPEEYELAKM LTRKGSLFQN ILAKRPFFTK EGYGSIKKIY | |
| | VWTDQDEIFL PEFQLWQIEN YKPDKVYKVE GGDHKLQLTK | |
| | TKEIAEILQE VADTYN | |
| SEQ.ID.NO. 17 | MVSAHFILIH TICHGAWLWY KLIPLLQSAG HNATAIDLVA | Baliospermum |
| | SGIDPRQLEQ IGTWEQYSEP LFTLIESIPE GKKVILVGEA | montanum |
| | GGGINIALAA EKYPEKVSAL VFHNALMPDI DHSPAFVYKK | |
| | FSEVFTDWKD SIFSNYTYGN DTVTAVELGD RTLAENIFSN | |
| | SPIEDVELAK HLVRKGSFFE QDLDTLPNFT SEGYGSIRRV | |
| | YVYGEEDQIF SRDFQLWQIN NYKPDKVYCV PSADHKIQIS | |
| | KVNELAQILQ EVANSASDLL AVA | |
| SEQ.ID.NO. 18 | MVTAHFVLIHTICHG | Cassava |
| SEQ.ID.NO. 19 | MLVLFISLLALTRPAMG | Cassava |

1-94. (canceled)

95. A transgenic cassava plant, comprising a heterologous nucleic acid sequence comprising a promoter preferentially active in a root that is operatively linked to a nucleic acid sequence encoding a storage protein.

96. The transgenic cassava plant of claim 95, wherein said storage protein is targeted to apoplasts of cells of said transgenic cassava plant.

97. The transgenic cassava plant of claim 95, wherein said storage protein is selected from the group consisting of hydroxynitrile lyases, arachins, avenins, cocosins, conarchins, concocosins, conglutins, conglycinins, convicines, crambins, cruciferins, cucurbitins, dioscorins, edestins, excelesins, gliadins, glutens, glytenins, glycinins, helianthins, hordeins, kafirins, legumins, napins, oryzins, pennisetins, phaseolins, prolamines, psophocarpins, secalins, sporamins, tryspsin inhibitors, vicilins, vicines, and zeins.

98. The transgenic cassava plant of claim 95, wherein said promoter is a potato class I patatin promoter.

99. The transgenic cassava plant of claim 98, wherein said promoter has at least 80% nucleotide sequence identity to class I patatin promoter having the nucleotide sequence shown in SEQ ID NO:13.

100. A method of increasing the protein content of a plant that produces cyanogenic glucosides, comprising:
i) transforming said plant with at least one nucleic acid molecule comprising a promoter preferentially active in a root or storage organ operatively linked to at least one transgene that encodes a storage protein, to produce a transgenic plant;
ii) selecting said transgenic plant comprising said at least one transgene; and
iii) growing said transgenic plant to produce a plant exhibiting increased protein content when compared to an equivalent, non-transgenic plant grown under similar conditions.

101. The method of claim 100, wherein said storage protein is targeted to apoplasts of cells of said transgenic plant.

102. The method of claim 100, wherein said storage protein is selected from the group consisting of hydroxynitrile lyases, arachins, avenins, cocosins, conarchins, concocosins, conglutins, conglycinins, convicines, crambins, cruciferins, cucurbitins, dioscorins, edestins, excelesins, gliadins, glutens, glytenins, glycinins, helianthins, hordeins, kafirins, legumins, napins, oryzins, pennisetins, phaseolins, prolamines, psophocarpins, secalins, sporamins, tryspsin inhibitors, vicilins, vicines, and zeins.

103. The method of claim 100, wherein said transgenic plant is selected from the group consisting of transgenic Cassava (Manihot esculenta), transgenic Sorghum (Sorghum vulgare), transgenic Flax (Linum usitatissimum), transgenic Lima beans (Phaseolus lunatus), transgenic Giant taro (Alocasia macrorrhizos), transgenic Bamboo (Bambusa arundinacea), transgenic Apple (Malus spp.), transgenic Peach (Prunus persica), transgenic Nectarine (Prunus persica var nucipersica), transgenic Cherry (Prunus spp.), transgenic Bitter almond (Prunus dulcis), transgenic raspberry, and transgenic crabapple.

104. The method of claim 100, wherein said promoter is a potato class I patatin promoter.

105. The method of claim 100, wherein said promoter has at least 80% nucleotide sequence identity to class I patatin promoter having the nucleotide sequence shown in SEQ ID NO:13.

106. A transgenic plant, or part or progeny thereof, produced by the method of claim 100.

107. A method of decreasing the cyanide content of a plant that produces cyanogenic glucosides, comprising:
i) transforming said plant with at least one nucleic acid molecule comprising a promoter preferentially active in a root or storage organ operatively linked to at least one transgene that encodes a plant hydroxynitrile lyase, to produce a transgenic plant;
ii) selecting said transgenic plant comprising said at least one transgene; and
iii) growing said transgenic plant to produce a plant exhibiting decreased cyanide content when compared to an equivalent non-transgenic plant grown under similar conditions,
wherein said promoter drives expression of said hydroxynitrile lyase substantially exclusively in roots or tubers of said transgenic plant.

108. The method of claim 107, wherein said hydroxynitrile lyase is targeted to apoplasts of cells of said transgenic plant.

109. The method of claim 107, wherein said transgenic plant is selected from the group consisting of transgenic Cassava (Manihot esculenta), transgenic Sorghum (Sorghum vulgare), transgenic Flax (Linum usitatissimum), transgenic Lima beans (Phaseolus lunatus), transgenic Giant taro (Alocasia macrorrhizos), transgenic Bamboo (Bambusa arundinacea), transgenic Apple (Malus spp.), transgenic Peach (Prunus persica), transgenic Nectarine (Prunus persica var nucipersica), transgenic Cherry (Prunus spp.), transgenic Bitter almond (Prunus dulcis), transgenic raspberry, and transgenic crabapple.

110. The method of claim 107, wherein said promoter is a potato class I patatin promoter.

111. The method of claim 107, wherein said promoter has at least 80% nucleotide sequence identity to class I patatin promoter having the nucleotide sequence shown in SEQ ID NO:13.

112. A transgenic plant, or part or progeny thereof, produced by the method of claim 107.


SEQUENCE LISTING (19 sequences)

SEQ ID NO: 1 - DNA, 37 bp, Artificial Sequence
  PCR Primer
gagactgcagttgtagttaatgcgtattagttttagc

SEQ ID NO: 2 - DNA, 36 bp, Artificial Sequence
  PCR Primer
tctcggtaccgatctagtaacatagatgacaccgcg

SEQ ID NO: 3 - DNA, 20 bp, Artificial Sequence
  PCR Primer
gtggaggaactggttctgga

SEQ ID NO: 4 - DNA, 20 bp, Artificial Sequence
  PCR Primer
tgcactcatctgcattctcc

SEQ ID NO: 5 - DNA, 20 bp, Artificial Sequence
  PCR Primer
caaaccagcccttgagagag

SEQ ID NO: 6 - DNA, 20 bp, Artificial Sequence
  PCR Primer
ttccccttgagggagtttct

SEQ ID NO: 7 - DNA, 19 bp, Artificial Sequence
  PCR Primer
aagctcaaaccagcccttg

SEQ ID NO: 8 - DNA, 20 bp, Artificial Sequence
  PCR Primer
aatttgccagcgttgaaagt

SEQ ID NO: 9 - DNA, 24 bp, Artificial Sequence
  PCR Primer
cgtctcacaaaatttttagtgacg

SEQ ID NO: 10 - DNA, 27 bp, Artificial Sequence
  PCR Primer
tgatgtttattatctcactcactttgc

SEQ ID NO: 11 - DNA, 24 bp, Artificial Sequence
  PCR Primer
cacatcaatccacttgctttgaag

SEQ ID NO: 12 - DNA, 19 bp, Artificial Sequence
  PCR Primer
catggtggagcacgacact

SEQ ID NO: 13 - DNA, 999 bp, Solanum tuberosum
ttgtagttaatgcgtattagttttagcgacgaagcactaaatcgtctttgtatactttgagtgacacatgtttagtgacgactgattgacgaaatttttttcgtctcacaaaatttttagtgacgaaacatgatttatagatgacgaaattatttgtccctcataatctaatttgttgtagtgatcattactcctttgtttgttttatttgtcatgttagttcattaaaaaaaaaatctctcttcttatcaatcctgacgtgtttaatatcataagattaaaaaatattttaatatatctttaatttaaactcacaaagtttaattttcttcgttaacttaatttgtcaaatcaggctcaaagatcgtttttcatatcggaatgaggattttatttattcttttaaaaataaagaggtgttgagctaaacaatttcaaatctcatcacacatatggggtcagccacaaaaataaagaacggttggaacggatctattatataatactaataaagaatagaaaaaggaaagtgagtgaggtgcgagggagagaatctgtttaatatcagagtcgatcatgtgtcagttttatcgatatgactttgacttcaactgagtttaagcaattctgataaggcgaggaaaatcacagtgctgaatctagaaaaatcttatacaatgtgagataaatctcaacaaaaacgttgagtccatagagggggtgtatgtgacaccccaacctcagcaaaagaaaacctcccctcaagaaggacatttgcggtgctaaacaatttcaagtctcatcacacatatatattatataatactaataaagaatagaaaaaggaaaggtaaacatcactaatgacagttgcggtgcaaagtgagtgagataataaacatcagtaatagacatcactaacttttattggttatgtcaaactcaaaataaaatttctcaacttgtttacgtgcctatatataccatgcttgttatatg

SEQ ID NO: 14 - protein, 9 aa, Manihot esculenta
  MISC_FEATURE (2)..(2): Xaa can be any naturally occuring amino acid
GXSCXGAGA

SEQ ID NO: 15 - protein, 258 aa, Manihot esculenta
MVTAHFVLIHTICHGAWIWHKLKPALERAGHKVTALDMAASGIDPRQIEQINSFDEYSEPLLTFLEKLPQGEKVIIVGESCAGLNIAIAADRYVDKIAAGVFHNSLLPDTVHSPSYTVEKLLESLPDWRDTEYFTFTNITGETITTMKLGFVLLRENLFTKCTDGEYELAKMVMRKGSLFQNVLAQRPKFTEKGYGSIKKVYIWTDQDKVFLPDFQRWQIANYKPDKAYQVQGGDHKLQLTKTEEVAHILQEVADAYA

SEQ ID NO: 16 - protein, 257 aa, Hevea brasiliensis
MAFAHFVLIHTICHGAWIWHKLKPLLEALGHKVTALDLAASGVDPRQIEEIGSFDEYSEPLLTFLEALPPGEKVILVGESCGGLNIAIAADKYCEKIAAAVFHNSVLPDTEHCPSYVVDKLMEVFPDWKDTTYFTYTKDGKEITGLKLGFTLLRENLYTLCGPEEYELAKMLTRKGSLFQNILAKRPFFTKEGYGSIKKIYVWTDQDEIFLPEFQLWQIENYKPDKVYKVEGGDHKLQLTKTKEIAEILQEVADTYN

SEQ ID NO: 17 - protein, 263 aa, Baliospermum montanum
MVSAHFILIHTICHGAWLWYKLIPLLQSAGHNATAIDLVASGIDPRQLEQIGTWEQYSEPLFTLIESIPEGKKVILVGEAGGGINIALAAEKYPEKVSALVFHNALMPDIDHSPAFVYKKFSEVFTDWKDSIFSNYTYGNDTVTAVELGDRTLAENIFSNSPIEDVELAKHLVRKGSFFEQDLDTLPNFTSEGYGSIRRVYVYGEEDQIFSRDFQLWQINNYKPDKVYCVPSADHKIQISKVNELAQILQEVANSASDLLAVA

SEQ ID NO: 18 - protein, 15 aa, Manihot esculenta
MVTAHFVLIHTICHG

SEQ ID NO: 19 - protein, 17 aa, Manihot esculenta
MLVLFISLLALTRPAMG